SEQUENCE LISTING (34 sequences)

SEQ ID NO: 1 - protein, 8 aa, Homo sapiens
KWKLFKKI

SEQ ID NO: 2 - protein, 8 aa, Homo sapiens
KWKVFKKI

SEQ ID NO: 3 - protein, 8 aa, Homo sapiens
RWKIFKKI

SEQ ID NO: 4 - protein, 10 aa, Homo sapiens
SWLSKTAKKL

SEQ ID NO: 5 - protein, 29 aa, Homo sapiens
RGLRRLGRKIAHGVKKYGPTVLRIIRIAG

SEQ ID NO: 6 - protein, 37 aa, Homo sapiens
LLGDFFRKSKEKIGKEFKRIVQRIKDFLRNLVPRTES

SEQ ID NO: 7 - protein, 23 aa, Homo sapiens
GIGKFLHSAKKFGKAFVGEIMNS

SEQ ID NO: 8 - protein, 13 aa, Homo sapiens
ILPWKWPWWPWRR

SEQ ID NO: 9 - protein, 185 aa, Acinetobacter baumannii
MILTKDGFSIIRNELFGGKLDQTQVDAINFIVAKATESGLTYPEAAYLLATIYHETGLPSGYRTMQPIKEAGSDSYLRSKKYYPYIGYGYVQLTWKENYERIGKLIGVDLIKNPEKALEPLIAIQIAIKGMLNGWFTGVGFRRKRPVSKYNKQQYVAARNIINGKDKAELIAKYAIIFERALRSL

SEQ ID NO: 10 - protein, 185 aa, Acinetobacter baumannii; identical to SEQ ID NO: 9

SEQ ID NO: 11 - protein, 146 aa, Acinetobacter baumannii
MQTSDKGIALIKEFEGCKLTAYQDSVGVWTIGYGWTQPVDGKPIRAGMTIKQETAERLLKTGMVSYESDVSRLVKVGLTQGQFDALVSFTYNLGARSLSTSTLLRKLNAGDYAGAADEFLRWNKAGGKVLNGLTRRREAERALFLS

SEQ ID NO: 12 - protein, 170 aa, Acinetobacter baumannii
MNFDKAFDTTIGHEGGFTLNKNDAGNWTGGKVGVGQLKGTKYGIAANSYPNLDIKNLTLDQAKAIYKRDYWDKAKCDLLPEGLKFHVFDVAVNSGVSRGIKTLQQAAGVKDDGIIGPNTLAAIKSFDESELLLRFYSFRISFYTSLSSFSNFGKGWMNRVANNLKLGTGG

SEQ ID NO: 13 - protein, 185 aa, Acinetobacter baumannii
MILTKDGFGIIRNELFGGKLDQTQVDAINFIVEKATESGLSYPEAAYLLATIYHETGLPSGYRTMQPIKEAGSDNYLRSKKYYPYIGYGYVQLTWKENYGRIGKLIGIDLIKNPEKALEPLIAIQIAIKGMLNGWFTGVGFRRKRPVSKYNKQQYIAARNIINGKDKAELIAKYAIIFERALRSL

SEQ ID NO: 14 - DNA, 555 bp, Acinetobacter baumannii
atgattctgactaaagacggatttagtattatccgtaatgaactattcggtggtaagttagatcaaactcaagtagatgcgataaactttattgtagcgaaggctactgagtctggtttaacctatccagaggcagcttatttactagctactatttaccatgagactggtttaccaagtggctatcgaactatgcaacctattaaagaagctggttctgatagctatcttcggtctaagaagtattacccttacatcggttatggttatgtacagttaacttggaaggagaactatgaacgtattggtaaacttattggagttgatctaattaagaatcccgaaaaggcactagaaccattaattgctattcagattgctatcaaaggtatgttgaatggttggttcacaggtgttgggttcagacgtaaacgtccagttagtaagtacaacaaacagcagtacgtagctgctcgtaatatcattaatgggaaagataaggctgagcttatagcgaagtacgctattatctttgaacgtgctctacggagctta

SEQ ID NO: 15 - DNA, 555 bp, Acinetobacter baumannii
atgattctgactaaagacgggtttagtattatccgtaatgaactattcggaggtaagttagatcaaactcaagtagatgcgataaactttattgtagagaaagctactgagtctggtttatcttatccagaggcagcctatttactagctaccatttatcatgagactggtttaccaagtggttatcgaactatgcaaccgattaaagaagctggttcggatagctaccttcgatctaagaagtactacccttacattggttatggttatgtgcagttaacttgggaggagaattatggacgtattagtaaacttattggagttgacctgattaagaatcctgagaaagccctagaacctttaattgctattcagattgctatcaaaggtatgttgaatggttggttcacaggtgttggattccgacgtaaacgtccagttagtaaatacaacaaacagcagtacgtagctgctcgtaatatcattaatgggaaagataaggctgagcttatagcgaagtacgctattatctttgaacgcgctctacggagctta

SEQ ID NO: 16 - DNA, 438 bp, Acinetobacter baumannii
atgcaaaccagtgataaaggcattgccctgatcaaagagttcgaaggttgcaagctcactgcctatcaggacagcgtcggcgtttggacgatcggttatggctggactcagcccgtcgacgggaaaccaatccgtgccgggatgacaattaagcaggaaacggcagaacgcctgctgaagacaggaatggtcagttacgaaagtgacgtgtcacgactggttaaagttggcctgactcaggggcaattcgatgccctggtgtcgttcacgtataacctcggtgcgcggtcattgtcgacatcgactctcctgcgaaaactcaacgccggagattacgctggtgcagccgatgagttcctgcgctggaataaagctggtgggaaggtgctgaatgggctgacacgtcggcgggaggcagagcgagctctgttcctgtca

SEQ ID NO: 17 - DNA, 510 bp, Acinetobacter baumannii
atgaactttgacaaagcatttgatacgactatcggccatgagggcgggtttacactcaataaaaacgatgctggcaactggacaggcggtaaagttggagttggtcagttaaagggcactaagtatggaattgctgcaaatagttatccaaatctggacattaaaaaccttactcttgatcaagcaaaggcaatttataagcgcgattattgggataaggcaaaatgcgatttattaccagaaggcttgaaattccatgtttttgatgtagctgtgaatagtggcgttagtcgtggcatcaagacacttcagcaagctgctggtgttaaagatgatggtattatcggccctaatacattagcagccataaagtcatttgatgaaagtgaattgctattaaggttctattcttttcgtatttctttttacacgtcattaagctcattctctaattttggcaaagggtggatgaatcgtgttgcgaataatcttaaacttggcactggcggt

SEQ ID NO: 18 - DNA, 555 bp, Acinetobacter baumannii
atgattctgactaaagatgggtttggtattatccgtaatgaactattcggaggtaagttagatcaaactcaagtagatgcaataaactttattgtagagaaagctactgagtctggtttatcttatccagaggcagcctatttactagctaccatctatcatgagactggtctaccaagcggttatcgaactatgcaacctattaaagaagctggttctgataactaccttcgatctaagaagtactacccgtacattggttatggttatgtacagttaacttggaaggagaactatggacggattggtaaacttattggaattgacctaattaagaatcctgagaaagcgctagaacctttaattgctattcagattgctatcaaaggcatgttgaatggttggttcacaggtgttggattccgacgtaaacgtccagttagtaaatacaacaaacagcagtacatagctgcgcgtaatatcattaatgggaaagataaggctgagcttatagcgaagtacgctattatctttgaacgcgctctacggagctta

SEQ ID NO: 19 - protein, 5 aa, Artificial Sequence
  linker sequence
GGSGG

SEQ ID NO: 20 - protein, 23 aa, Artificial Sequence
  CeA peptide octamer and C-terminal region of LysAB2/PlyAB1
IIFERALRSLGGSGGKWKLFKKI

SEQ ID NO: 21 - DNA, 35 bp, Artificial Sequence
  nucleotide primer
tatgaaatggaaactgttcaagaaaatcggttccg

SEQ ID NO: 22 - DNA, 37 bp, Artificial Sequence
  nucleotide primer
gatccggaaccgattttcttgaacagtttccatttca

SEQ ID NO: 23 - DNA, 48 bp, Artificial Sequence
  nucleotide primer
agcttggtggctctggtggcaaatggaaactgttcaagaaaatctgac

SEQ ID NO: 24 - DNA, 48 bp, Artificial Sequence
  nucleotide primer
tcgagtcagattttcttgaacagtttccatttgccaccagagccacca

SEQ ID NO: 25 - DNA, 27 bp, Artificial Sequence
  nucleotide primer
cgcggatccatgattctgactaaagac

SEQ ID NO: 26 - DNA, 27 bp, Artificial Sequence
  nucleotide primer
gtgctcgagtcagattttcttgaacag

SEQ ID NO: 27 - DNA, 49 bp, Artificial Sequence
  nucleotide primer
caagtggctatcgaactatgcaacctattaaagaagctggttctgatag

SEQ ID NO: 28 - DNA, 49 bp, Artificial Sequence
  nucleotide primer
gcatagttcgatagccacttggtaaaccagtcgcatggtaaatagtagc

SEQ ID NO: 29 - protein, 200 aa, Artificial Sequence
  recombinant lysin
MILTKDGFSIIRNELFGGKLDQTQVDAINFIVAKATESGLTYPEAAYLLATIYHETGLPSGYRTMQPIKEAGSDSYLRSKKYYPYIGYGYVQLTWKENYERIGKLIGVDLIKNPEKALEPLIAIQIAIKGMLNGWFTGVGFRRKRPVSKYNKQQYVAARNIINGKDKAELIAKYAIIFERALRSLKLGGSGGKWKLFKKI

SEQ ID NO: 30 - protein, 203 aa, Artificial Sequence
  recombinant lysin
MILTKDGFSIIRNELFGGKLDQTQVDAINFIVEKATESGLSYPEAAYLLATIYHETGLPSGYRTMQPIKEAGSDSYLRSKKYYPYIGYGYVQLTWEENYGRISKLIGVDLIKNPEKALEPLIAIQIAIKGMLNGWFTGVGFRRKRPVSKYNKQQYVAARNIINGKDKAELIAKYAIIFERALRSLEFGKLGGSGGKWKLFKKI

SEQ ID NO: 31 - protein, 206 aa, Artificial Sequence
  recombinant lysin
MILTKDGFGIIRNELFGGKLDQTQVDAINFIVEKATESGLSYPEAAYLLATIYHETGLPSGYRTMQPIKEAGSDNYLRSKKYYPYIGYGYVQLTWKENYGRIGKLIGIDLIKNPEKALEPLIAIQIAIKGMLNGWFTGVGFRRKRPVSKYNKQQYIAARNIINGKDKAELIAKYAIIFERALRSLEFGGSGKLGGSGGKWKLFKKI

SEQ ID NO: 32 - DNA, 615 bp, Artificial Sequence
  nucleotide sequence encoding recombinant lysin
ggatccatgattctgactaaagacggatttagtattatccgtaatgaactattcggtggtaagttagatcaaactcaagtagatgcgataaactttattgtagcgaaggctactgagtctggtttaacctatccagaggcagcttatttactagctactatttaccatgagactggtttaccaagtggctatcgaactatgcaacctattaaagaagctggttctgatagctatcttcggtctaagaagtattacccttacatcggttatggttatgtacagttaacttggaaggagaactatgaacgtattggtaaacttattggagttgatctaattaagaatcccgaaaaggcactagaaccattaattgctattcagattgctatcaaaggtatgttgaatggttggttcacaggtgttgggttcagacgtaaacgtccagttagtaagtacaacaaacagcagtacgtagctgctcgtaatatcattaatgggaaagataaggctgagcttatagcgaagtacgctattatctttgaacgtgctctacggagcttaaagcttggtggctctggtggcaaatggaaactgttcaagaaaatctgactcgag

SEQ ID NO: 33 - DNA, 624 bp, Artificial Sequence
  nucleotide sequence encoding recombinant lysin
ggatccatgattctgactaaagacgggtttagtattatccgtaatgaactattcggaggtaagttagatcaaactcaagtagatgcgataaactttattgtagagaaagctactgagtctggtttatcttatccagaggcagcctatttactagctaccatttatcatgagactggtttaccaagtggttatcgaactatgcaaccgattaaagaagctggttcggatagctaccttcgatctaagaagtactacccttacattggttatggttatgtgcagttaacttgggaggagaattatggacgtattagtaaacttattggagttgacctgattaagaatcctgagaaagccctagaacctttaattgctattcagattgctatcaaaggtatgttgaatggttggttcacaggtgttggattccgacgtaaacgtccagttagtaaatacaacaaacagcagtacgtagctgctcgtaatatcattaatgggaaagataaggctgagcttatagcgaagtacgctattatctttgaacgcgctctacggagcttagaattcggtaagcttggtggctctggtggcaaatggaaactgttcaagaaaatctgactcgag

SEQ ID NO: 34 - DNA, 633 bp, Artificial Sequence
  nucleotide sequence encoding recombinant lysin
ggatccatgattctgactaaagatgggtttggtattatccgtaatgaactattcggaggtaagttagatcaaactcaagtagatgcaataaactttattgtagagaaagctactgagtctggtttatcttatccagaggcagcctatttactagctaccatctatcatgagactggtctaccaagcggttatcgaactatgcaacctattaaagaagctggttctgataactaccttcgatctaagaagtactacccgtacattggttatggttatgtacagttaacttggaaggagaactatggacggattggtaaacttattggaattgacctaattaagaatcctgagaaagcgctagaacctttaattgctattcagattgctatcaaaggcatgttgaatggttggttcacaggtgttggattccgacgtaaacgtccagttagtaaatacaacaaacagcagtacatagctgcgcgtaatatcattaatgggaaagataaggctgagcttatagcgaagtacgctattatctttgaacgcgctctacggagcttagaattcggtggatctggtaagcttggtggctctggtggcaaatggaaactgttcaagaaaatctgactcgag

(19) United States
(12) Patent Application Publication
(10) Pub. No.: US 2022/0227817 A1
(43) Pub. Date: Jul. 21, 2022

(54) ENGINEERED GLOBULAR ENDOLYSIN, A HIGHLY POTENT ANTIBACTERIAL ENZYME FOR MULTIDRUG RESISTANT GRAM-NEGATIVE BACTERIA

(71) Applicant: The Chinese University of Hong Kong, Hong Kong (CN)

(72) Inventors: Jiang XIA, Hong Kong (CN); Shui Yee, Sharon LEUNG, Hong Kong (CN); Xi CHEN, Shenzhen (CN); Miao LIU, Tianjin (CN)

(21) Appl. No.: 17/648,655

(22) Filed: Jan. 21, 2022

Related U.S. Application Data
(60) Provisional application No. 63/139,846, filed on Jan. 21, 2021.

(51) Int. Cl. C07K 14/005; A61P 31/04; C12N 9/36

(52) U.S. Cl. CPC C07K 14/005; C07K 2319/33; C12N 9/2462; A61P 31/04

(57) ABSTRACT

The subject invention pertains to lysins fused to a CeA peptide fragment, particularly at the C-terminus of the lysin. The subject invention also pertains to recombinant DNA encoding said lysins, vectors encoding said recombinant DNA, host cells comprising said vectors, and compositions comprising said lysins. The invention further pertains to a method of treating a bacterial infection, particularly a Gram-negative bacterial infection.

### CROSS-REFERENCE TO RELATED APPLICATION

This application claims the benefit of U.S. Patent Application Ser. No. 63/139,846, filed Jan. 21, 2021, which is hereby incorporated by reference in its entirety including any tables, figures, or drawings.

### BACKGROUND OF THE INVENTION

The emergence of antimicrobial resistance poses a great threat to the global health. Currently, infections caused by multidrug resistant bacteria lead to about 700,000 deaths per year, which can escalate to 10 million deaths annually, with a projected cost of $100 trillion by 2050 [1]. Antibacterial treatments with novel mechanisms that are different from those of currently available antibiotics are urgently needed to fight against drug resistant bacterial strains. Gram-negative bacteria pose a serious threat. The World Health Organization (WHO) published its first global priority pathogen list, in which nine out of the twelve identified pathogens are Gram-negative bacteria [2]. Alarmingly, outbreaks caused by Gram-negative bacteria Acinetobacter baumannii, Pseudomonas aeruginosa, and Klebsiella pneumoniae have been increasingly reported [3,4]. Efforts to develop novel antibacterial compositions against Gram-negative bacteria using novel mechanisms are critical.

The peptidoglycan (PG) degrading enzymes, endolysins (lysins), encoded by bacteriophages to lyse host bacterial cells at the end of the phage cycle have recently emerged as a promising class of novel antibacterial compositions; they are particularly effective against the Gram-positive bacteria [5-8]. Multiple lysins against Gram-positive bacteria have entered into clinical trials, and the anti-staphylococcal lysin, CF-301, developed by ContraFect (Yonkers, N.Y.) has been granted a Fast Track Designation to speed up the development process [9]. In contrary, the development of lysins against Gram-negative bacteria is lagging. The outer membrane (OM) of the Gram-negative bacterial cell wall forms a barrier for lysins to access and degrade the PG layer, rendering the lysin treatment ineffective against Gram-negative bacteria [10-13]. Increasing attention has been devoted to overcoming this barrier; viable methods to assist lysins to penetrate the OM include co-administration with chemical reagents, known as outer membrane permeabilizers (OMPs) that can compromise the OMP, such as EDTA or citric acid, encapsulation into carrier systems for outer membrane penetration, or modification of lysins by protein engineering. These approaches and an additional proposed method that includes protein engineering by fusing the native lysins with a membrane-penetrating peptide may be a promising strategy against Gram-negative bacteria [14].

There are preceding successes in engineering lysins against Gram-negative bacteria. Briers et al. fused modular lysins with membrane-penetrating peptides and developed engineered lysins called “artilysin” [15-18]. The polycationic nonapeptide was identified as a promising membrane-penetrating peptide due to its strong interaction with the negatively charged surface lipopolysaccharide (LPS). The fusion enzymes of two modular endolysins OBPgp279 and PVP-SE1gp146 could enhance the bactericidal activity by 2.6-log and 4 to 5-log reduction of the bacterial counts in the absence and presence of EDTA, respectively [15]. The same group also fused the SMAP-29 peptide at the N-terminal to the modular lysin KZ144 and found this engineered Art-175 lysin showed potent bactericidal activity and achieved complete eradication of the stationary phase bacteria (8-log reduction) in the presence of 0.5 mM EDTA [17]. The modular OBPgp279 lysin was also modified by Yang et al. by combining the Cecropin A (CeA) peptide residues 1-8 at the N-terminus and achieved potent activity against bacteria in the log growth phase (4 to 5-log reduction) and bacteria in the stationary growth phase (0.5 to 2-log killing) without OMPs [19]. The killing efficiency against stationary phase bacteria was increased up to 5-log in the presence of OMPs.

Notwithstanding these successes, most of the research focused on modular lysins that contain a C-terminus, enzymatically active domain and an N-terminal PG binding domain. For lysins targeting Gram-negative bacteria, most of them fall within the globular lysin category, which have a globular structure with only a single enzymatically active domain [10]. These Gram-negative lysins usually show no antibacterial activity when apply exogenously, except for a few with a cationic or amphipathic C-terminal peptide that can interact with the negatively charged LPS on the outer membrane [12]. Despite some intrinsic membrane-penetrating capacity, their antibacterial activities were only modest (<2-log reduction) [20-23].

Accordingly, a lysin with membrane-penetrating capabilities is needed.

### BRIEF SUMMARY OF THE INVENTION

The disclosure provides lysins fused to a CeA peptide fragment, particularly at the C-terminus of the lysin. The subject invention also pertains to recombinant DNA encoding said lysins, vectors encoding said nucleotides, host cells comprising said vectors, and compositions comprising said lysins. The disclosure further provides antimicrobial compositions that target Gram-negative bacteria. The compositions comprise a globular lysin that can exhibit high activity towards multidrug resistant Gram-negative bacteria. The compositions have numerous advantages, including long shelf-lives, high serum stability, and minimal toxicity towards mammalian cells. The disclosure further provides methods for treating bacterial infections using the compositions.

In certain embodiments, the outer membrane permeability of a Gram-negative bacterium is increased with the addition of a C-terminal sequence to a globular lysin. In certain embodiments, a globular lysin with only modest antibacterial activity, LysAB2, can be fused to a CeA peptide to increase the outer membrane permeability and antibacterial activity of a globular lysin to Gram-negative bacteria. The subject invention can be used to produce a highly potent engineered lysin enzyme variant that holds promise for future clinical use.

### BRIEF DESCRIPTION OF THE DRAWINGS

FIGS. 1A to 1E show design and antibacterial activities of a C-terminal engineered globular lysin LysAB2. FIG. 1A Schematic of LysAB2, the C-terminal and N-terminal modified constructs. FIG. 1B Antibacterial activity of the variants of LysAB2. Briefly, 8 μM of each lysin was incubated with logarithmic A. baumannii at 37° C. for 2 h in PBS. The colony forming units (CFUs) of the bacterial cultures after treatment were counted as a measurement for the antibacterial activity. FIG. 1C Dose-dependent antibacterial activity of LysAB2 and LysAB2-KWK against log phase A. baumannii. Cells were incubated with lysin in PBS buffer for 2 h before the numbers were counted. FIG. 1D Time-dependent inhibition curve (8 μM lysins). FIG. 1E Representative Scanning Electron Microscopy (SEM) images of the lysin-treated cells. Log phase A. baumannii cells were incubated with 8 μM enzymes before SEM imaging. Scale bar, 3 m.

FIG. 2 shows antibacterial activity of LysAB2-KWK against different Gram-negative and Gram-positive bacteria. 8 μM enzymes were incubated with different logarithmic phase bacteria in PBS at 37° C. for 2 h.

FIGS. 3A to 3C show antibacterial activity against stationary phase bacteria and antibiofilm activity of LysAB2-KWK. FIG. 3A shows antibacterial activity of 10 μM LysAB2 and LysAB2-KWK incubate with A. baumannii in logarithmic and stationary phase at 37° C. for 2 h in PBS. FIGS. 3B and 3C show LyAB2-KWK disrupted the A. baumannii biofilm. Biofilm formation of A. baumannii strain was growed in 96-well plates for 24 h, and then the biofilm was treated with PBS or 1-10M LysAB2-KWK at for 10 h. The residual biofilm was assessed by crystal violet staining (FIG. 3B) and viable cell counting (FIG. 3C).

FIGS. 4A to 4C show serum activity, cytotoxicity towards human cells and storage stability of LysAB2 KWK. FIG. 4A shows antibacterial activity of lysins in presence of human serum against A. baumannii. log phase bacteria were incubated with 12 μM of lysins in PBS at 37° C. for 2 h. FIG. 4B shows cell viability of B3ESA-2B cell after treated with lysins for 37° C. for 12 h. The effect of lysins on the viability of the cells was determined with CKK8 assay. FIG. 4C shows antibacterial activity of LysA32-KWK keeping at 4° C. for specific days against A. baumannii. Log phase bacteria were incubated with 8 μM of lysins in PBS at 37° C. for 2 h.

FIGS. 5A to 5C show mechanistic studies using LysAB2, LysAB2-KWK and the E55A mutant. FIG. 5A Muralytic activities of lysins characterized by turbidity on chloroform/Tris-HCl buffer treated A. baumannii cells. The concentrations of LysAB2, LysAB2-KWK and LysAB2-KWK E55A used in this assay was 4 nM, 4 nM and 1 μM respectively. FIG. 5B NPN uptake assay of A. baumannii induced by 8 μM lysins. Net fluorescence signals with the background signal of the cells subtracted are used. FIG. 5C Antibacterial activity of the lysins against log-phase A. baumannii. Cells were incubated with 8 μM enzymes in PBS at 37° C. for 15 min or 2 h.

FIGS. 6A to 6D show OM permeability and antibacterial activity of different peptide modified globular lysins. FIG. 6A NPN uptake of A. baumannii cells induced by four globular endolysins, and their C-terminal modifications. The fluorescence values have been subtracted by values of the cell. FIGS. 6B and 6C Antibacterial activity of four native and C-terminal modified endolysins. 15 μM of each lysin was incubated with logarithmic A. baumannii at 37° C. for 2 h in PBS. FIG. 6D Comparison of the sequences of lysins.

FIG. 7 shows antibacterial activity of four native and C-terminal modified endolysins. 15 μM of each lysin was incubated with logarithmic A. baumannii at 37° C. for 2 h in PBS.

FIG. 8 shows Time-killing curves of A. baumannii by different concentration of LysAB2-KWK.

FIG. 9 shows NPN uptake assay of A. baumannii induced by different concentration of polymyxin B. Net fluorescence signals with the background signal of the cells subtracted are used.

FIG. 10 shows analysis of purified proteins on 12% SDS-PAGE gel. M: marker; 1: LysAB2; 2: LysAB2 KWK; 3: PlyAB1; 4: PlyAB1 KWK; 5: PlyE146; 6: PlyE146 KWK; 7: 68 lysin; 8: 68 lysin KWK.

### BRIEF DESCRIPTION OF THE SEQUENCES

SEQ ID NO: 1: CeA peptide octamer

SEQ ID NO: 2: CeB peptide octamer

SEQ ID NO: 3: Papiliocin peptide octamer

SEQ ID NO: 4: Cecropin P1 peptide

SEQ ID NO: 5: SMAP-29 peptide

SEQ ID NO: 6: LL-37 peptide

SEQ ID NO: 7: Magainin II peptide

SEQ ID NO: 8: Indolicidin peptide

SEQ ID NO: 9: LysAB2 amino acid sequence

SEQ ID NO: 10: PlyAB1 amino acid sequence

SEQ ID NO: 11: PlyE146 amino acid sequence

SEQ ID NO: 12: 68 Lysin amino acid sequence

SEQ ID NO: 13: ABgp46 amino acid sequence

SEQ ID NO: 14: nucleotide sequence encoding LysAB2

SEQ ID NO: 15: nucleotide sequence encoding PlyAB1

SEQ ID NO: 16: nucleotide sequence encoding PlyE146

SEQ ID NO: 17: nucleotide sequence encoding 68 Lysin

SEQ ID NO: 18: nucleotide sequence encoding ABgp46 SEQ ID NO: 19: Exemplary amino acid linker sequence

SEQ ID NO: 20: CeA peptide octamer and C-terminal region of LysAB2/PlyAB1

SEQ ID NO: 21: Nucleotide primer sequence to create vector encoding KWK-LysAB2

SEQ ID NO: 22: Nucleotide primer sequence to create vector encoding KWK-LysAB2

SEQ ID NO: 23: Nucleotide primer sequence to create vector encoding LysAB2-KWK

SEQ ID NO: 24: Nucleotide primer sequence to create vector encoding LysAB2-KWK

SEQ ID NO: 25: Nucleotide primer sequence to create E55A mutation of LysAB2

SEQ ID NO: 26: Nucleotide primer sequence to create E55A mutation of LysAB2

SEQ ID NO: 27: Nucleotide primer sequence to create E55A mutation of LysAB2

SEQ ID NO: 28: Nucleotide primer sequence to create E55A mutation of LysAB2

SEQ ID NO: 29: LysAB2-KWK amino acid sequence

SEQ ID NO: 30: PlyAB1-KWK amino acid sequence

SEQ ID NO: 31: ABgp46-KWK amino acid sequence

SEQ ID NO: 32: nucleotide sequence encoding LysAB2-KWK

SEQ ID NO: 33: nucleotide sequence encoding PlyAB1-KWK

SEQ ID NO: 34: nucleotide sequence encoding ABgp46-KWK

### DETAILED DISCLOSURE OF THE INVENTION

The subject invention relates to novel antimicrobial agents. The agents can be used to inhibit the growth of Gram-negative bacteria or kill Gram-negative bacteria. In particular, the antimicrobial agents comprise a lysin fused to a peptide.

### Definitions

Ranges provided herein are understood to be shorthand for all of the values within the range. For example, a range of 1 to 20 is understood to include any number, combination of numbers, or sub-range from the group consisting 1, 2, 3, 4, 5, 6, 7, 8, 9, 10, 11, 12, 13, 14, 15, 16, 17, 18, 19 and 20, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

As used herein a “reduction” means a negative alteration, and an “increase” means a positive alteration, wherein the negative or positive alteration is at least 0.001%, 0.01%, 0.1%, 0.5%, 1%, 5%, 10%, 15%, 20%, 25%, 30%, 35%, 40%, 45%, 50%, 55%, 60%, 65%, 70%, 75%, 80%, 85%, 90%, 95% or 100%.

The transitional term “comprising,” which is synonymous with “including,” or “containing,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention. Use of the term “comprising” contemplates other embodiments that “consist” or “consist essentially of” the recited component(s).

Unless specifically stated or obvious from context, as used herein, the term “or” is understood to be inclusive. Unless specifically stated or obvious from context, as used herein, the terms “a,” “and” and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the term “fusion protein” refers to a translated protein product resulting from the expression of two fused nucleic acid sequences. Such a protein may be produced, for example, in recombinant DNA expression systems. Moreover, the term “fusion protein” as used herein refers to a fusion of a first amino acid sequence as e.g. an enzyme, with a second or further amino acid sequence. The second or further amino acid sequence may define a domain or any kind of peptide stretch. Preferably, said second and/or further amino acid sequence is foreign to and not substantially homologous with any domain of the first amino acid sequence.

As used herein, the terms “endolysin” or “lysin” as used herein refers to an enzyme which is suitable to hydrolyse bacterial cell walls. “Endolysins” or “lysins” can comprise at least one “enzymatically active domain” (EAD) having at least one of the following activities: endopeptidase, chitinase, T4 like muraminidase, lambda like muraminidase, N-acetyl-muramoyl-L-alanine-amidase (amidase), muramoyl-L-alanine-amidase, muramidase, lytic transglycosylase (C), lytic transglycosylase (M), N-acetyl-muramidase, N-acetyl-glucosaminidase (lysozyme) or transglycosylases as e.g. KZ144 and EL188. In addition, the endolysins may contain also regions which are enzymatically inactive, and bind to the cell wall of the host bacteria, the so-called CBDs (cell wall binding domains).

As used herein, the term “deletion” refers to the removal of at least 1, 2, 3, 4, 5, 6, 7, 8, 9, 10 or more amino acid residues from the respective starting sequence.

As used herein, the term “insertion” or “addition” refers to the insertion or addition of at least 1, 2, 3, 4, 5, 6, 7, 8, 9, 10 or more amino acid residues to the respective starting sequence.

As used herein, the term “substitution” refers to the exchange of an amino acid residue located at a certain position for a different one.

As used herein, “Gram-negative bacteria” generally refers to bacteria which produce a crystal violet stain that is decolorized in Gram staining, i. e. the cells do not retain crystal violet dye in the Gram staining protocol. As used herein, the term “Gram-negative bacteria” may describe without limitation one or more (i.e., one or a combination) of the following bacterial species: Acinetobacter baumannii, Acinetobacter haemolyticus, Actinobacillus actinomycetemcomitans, Aeromonas hydrophila, Bacteroides fragilis, Bacteroides theataioatamicron, Bacteroides distasonis, Bacteroides ovatus, Bacteroides vulgatus, Bordetella pertussis, Brucella melitensis, Burkholderia cepacia, Burkholderia pseudomallei, Burkholderia mallei, Prevotella corporis, Prevotella intermedia, Prevotella endodontalis, Porphyromonas asacchitrolytica, Campylobacter jejuni, Campylobacter coli, Campylobacter fetus, Citrobacter freundii, Citrobacter koseri, Edwarsiella tarda, Eikenella corrodens, Enterobacter cloacae, Enterobacter aerogeries, Enterobacter agglomerans, Escherichia coli, Francisella tularensis, Haemophilus influenzae, Haemophilus ducreyi, Helicobacter pylori, Kingella kingae, Klebsiella pneumoniae, Klebsiella oxytoca, Klebsiella rhinoscleromatis, Klebsiella ozaenae, Legionella pemimophila, Moraxella catarrhalis, Morganella morganii, Neisseria gonorrhoeae, Neisseria meningitidis, Pasteurella multocida, Plesiomonas shigelloides, Proteus mirabilis, Proteus vulgaris, Proteus penneri, Proteus myxofaciens, Providencia stuartii, Providencia rettgeri, Providencia alcalifaciens, Pseudomonas aeruginosa, Pseudomonas fluorescens, Salmonella typhi, Salmonella paratyphi, Serratia marcescens, Shigella flexneri, Shigella boydii, Shigella sonnei, Shigella dysenteriae, Stenotrophomonas maltophilia, Streptobacillus moniliformis, Vibrio cholerae, Vibrio parahaemolyticus, Vibrio vulificus, Vibrio alginolyticus, Yersinia enterocolitica, Yersinia pestis, Yersinia pseudotuberculosis, Chlamydophila pneumoniae, Chlamydophila trachomatis, Ricketsia prowazekii, Coxiella burnetii, Ehrlichia chaffeensis, or Bartonella hensenae. The compounds of the present disclosure will be useful in inhibiting pathogenic bacterial growth and in treating one or more bacterial infections, particularly but not necessarily exclusively involving Gram-negative bacteria.

As used herein, the term “bactericidal” in the context of an agent conventionally means having the property of causing the death of bacteria or capable of killing bacteria to an extent of at least a 3-log (99.9%) or better reduction among an initial population of bacteria.

As used herein, the term “bacteriostatic” conventionally means having the property of inhibiting bacterial growth, including inhibiting growing bacterial cells, thus causing a 2-log (99%) or better and up to just under a 3-log reduction among an initial population of bacteria.

As used herein, the term “antibacterial” in a context of an agent is used generically to include both bacteriostatic and bactericidal agents.

As used herein, the term “drug resistant” in a context of a pathogen and more specifically a bacterium, generally refers to a bacterium that is resistant to the antimicrobial activity of a drug. When used in a more particular way, drug resistance specifically refers to antibiotic resistance. In some cases, a bacterium that is generally susceptible to a particular antibiotic can develop resistance to the antibiotic, thereby becoming a drug resistant microbe or strain. A “multi-drug resistant” pathogen is one that has developed resistance to at least two classes of antimicrobial drugs, each used as monotherapy. For example, certain strains of Pseudomonas aeruginosa have been found to be resistant to nearly all or all antibiotics including aminoglycosides, cephalosporins, fluoroquinolones, and carbapenems (Antibiotic Resistant Threats in the United States, 2013, U.S. Department of Health and Services, Centers for Disease Control and Prevention). One skilled in the art can readily determine if a bacterium is drug resistant using routine laboratory techniques that determine the susceptibility or resistance of a bacterium to a drug or antibiotic.

As used herein, the term “pharmaceutically acceptable” means compatible with the other ingredients of a pharmaceutical composition and not deleterious to the recipient thereof.

As used herein, the phrase “percent amino acid sequence identity” with respect to the lysin polypeptide sequences is defined herein as the percentage of amino acid residues in a candidate sequence that are identical with the amino acid residues in the specific lysin polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for example, using publicly available software such as BLAST or Megalign (DNASTAR) software. Two or more polypeptide sequences can be anywhere from 0-100% identical, or any integer value there between. In the context of the present disclosure, two polypeptides are “substantially identical” when at least 80% of the amino acid residues (preferably at least about 85%, at least about 90%, and preferably at least about 95%) are identical. The term “percent (%) amino acid sequence identity” as described herein applies to lysin enzymes as well. Thus, the term “substantially identical” will encompass mutated, truncated, fused, or otherwise sequence-modified variants of isolated lysin polypeptides and peptides described herein, and active fragments thereof, as well as polypeptides with substantial sequence identity (e.g., at least 80%, at least 85%, at least 90%, or at least 95% identity as measured for example by one or more methods referenced above) as compared to the reference polypeptide.

Two amino acid sequences are “substantially homologous” when at least about 80% of the amino acid residues (preferably at least about 85%, at least about 90%, and preferably at least about 95%) are identical, or represent conservative substitutions. The sequences of lysin polypeptides of the present disclosure, are substantially homologous when one or more, or several, or up to 10%, or up to 15%, or up to 20% of the amino acids of the lysin polypeptide are substituted with a similar or conservative amino acid substitution, and wherein the resulting lysin have the profile of activities, antibacterial effects, and/or bacterial specificities of lysin polypeptides disclosed herein. The meaning of “substantially homologous” described herein applies to lysin enzymes as well.

As used herein, the term “subject” refers to a mammal, a plant, a lower animal, a single cell organism, or a cell culture. For example, the term “subject” is intended to include organisms, such as, for example, prokaryotes and eukaryotes, which are susceptible to or afflicted with bacterial infections, for example Gram-positive or Gram-negative bacterial infections. Examples of subjects include mammals, such as, for example, humans, dogs, cows, horses, pigs, sheep, goats, cats, mice, rabbits, rats, and transgenic non-human animals. In certain embodiments, the 11 subject is a human, such as, for example, a human suffering from, at risk of suffering from, or susceptible to infection by Gram-negative bacteria, whether such infection be systemic, topical or otherwise concentrated or confined to a particular organ or tissue.

### Engineered Lysins and Compositions Thereof

The present disclosure relates to novel antibacterial agents, particularly agents against Gram-negative bacteria. In particular, the present disclosure relates to lysin enzymes active against Gram-negative bacteria, such as Acinetobacter baumannii. Examples of such lysins are LysAB2, PlyAB1, PlyE146, 68 Lysin, and ABgp46, including polypeptides having an amino acid sequence within the set SEQ ID NO: 9, SEQ ID NO: 10, SEQ ID NO: 11, SEQ ID NO: 12, and SEQ ID NO: 13. Furthermore, in accordance with the present disclosure, such sequence modified peptides include fragments of the confirmed native Gram-negative lysin polypeptides maintaining lysin activity, as well as variants thereof having 80% or more (such as, for example, at least 85%, at least 90%, at least 95%, or at least 98%) sequence identity with the native lysin polypeptides or active fragments thereof; and, the nonidentical portions might include substitutions, additions, and/or deletions with both natural and non-natural (synthetic) amino acid residues.

In certain embodiments, the lysin is fused to a peptide. Preferably, the peptide of the fusion lysin protein is fused to the N-terminus and/or to the C-terminus of the lysin. In a particular preferred embodiment, said peptide is only fused to the C-terminus of the lysin enzyme. Said peptide on the N-terminus and on the C-terminus can be the same or distinct peptide stretches. The peptide stretch can be linked to the enzyme by additional amino acid residues e.g. due to cloning reasons. The said peptide stretch can be linked to the fusion lysin protein by at least 1, 2, 3, 4, 5, 6, 7, 8, 9 or 10 additional amino acid residues.

The peptide stretch of the fusion protein according to the present invention is preferably is encoded by nucleotides on a plasmid or the peptide stretch can be covalently bound to the lysin enzyme. Preferably, said peptide stretch consists of at least 5, more preferably at least of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or at least 100 amino acid residues. Especially preferred is a peptide stretch comprising about 5 to about 100 amino acid residues, about 5 to about 50 or about 5 to about 30 amino acid residues. More preferred is a peptide stretch comprising about 6 to about 42 amino acid residues, about 6 to about 39 amino acid residues, about 6 to about 38 amino acid residues, about 6 to about 31 amino acid residues, about 6 to about 25 amino acid residues, about 6 to about 24 amino acid residues, about 6 to about 22 amino acid residues, about 6 to about 21 amino acid residues, about 6 to about 20 amino acid residues, about 6 to about 19 amino acid residues, about 6 to about 16 amino acid residues, about 6 to about 14 amino acid residues, about 6 to about 12 amino acid residues, about 6 to about 10 amino acid residues, or about 6 to about 9 amino acid residues.

In certain embodiments, the peptide fused to a lysin is an antimicrobial peptide or a peptide fragment derived from an antimicrobial peptide. The peptide can be a defensin, such as, for example, Cathelicidine, Cecropin P1, Cecropin A (CeA), Cecropin B (CeB), Papiliocin, Cathelicidin, Indolicidin, or Magainin II. In certain embodiments, the peptide is CeA. In preferred embodiments, a portion of CeA is fused to a lysin. In more preferred embodiments, at least 4, 5, 6, 7, 8, 9, 10, 11, 12, or more amino acid residues derived from the can be fused to a lysin. The portion of CeA that is fused to a lysin can be SEQ ID NO: 1. Other examples of peptides can be SEQ ID NO: 2 (derived from CeB), SEQ ID NO: 3 (derived from Papiliocin), SEQ ID NO: 4 (derived Cecropin P1), SEQ ID NO: 5 (SMAP-29; derived from Cathelicidin), SEQ ID NO: 6 (LL-37, derived from Cathelicidin), SEQ ID NO: 7 (derived from Magainin II), and SEQ ID NO: 8 (derived from Indolicidin).

In certain embodiments, there may be other sequence elements present in the genetically-engineered lysins. The linking sequence can link the lysin to the peptide, including the antimicrobial peptide, which can be fused to the lysin. In preferred embodiments, the other sequence elements are short linker sequences not exceeding, for example 15, 14, 13, 12, 11, 10, 9, 8, 7, 6, 5, 4, 3, or 2 amino acids. In more preferred embodiments, the linker can be a flexible sequence comprising one or more glycine residues. An example of such a linker is a glycine-serine linker or the sequence GGSGG (SEQ ID NO: 19).

In certain embodiments, the lysin is specific for Gram-negative bacteria such as Gram-negative bacteria of bacterial groups, families, genera or species comprising strains pathogenic for humans or animals such as, for example, Enterobacteriaceae (Escherichia, especially E. coli, Salmonella, Shigella, Citrobacter, Edwardsiella, Enterobacter, Hafnia, Klebsiella, especially K. pneumoniae, Morganella, Proteus, Providencia, Serratia, Yersinia), Pseudomonadaceae (Pseudomonas, especially P. aeruginosa, Burkholderia, Stenotrophomonas, Shewanella, Sphingomonas, Comamonas), Neisseria, Moraxella, Vibrio, Aeromonas, Brucella, Francisella, Bordetella, Legionella, Bartonella, Coxiella, Haemophilus, Pasteurella, Mannheimia, Actinobacillus, Gardnerella, Spirochaetaceae (Treponema and Borrelia), Leptospiraceae, Campylobacter, Helicobacter, Spirillum, Streptobacillus, Bacteroidaceae (Bacteroides, Fusobacterium, Prevotella, Porphyromonas), Acinetobacter, especially A. baumannii.

In some embodiments, the present invention is directed to nucleic acid molecules, including recombinant DNA, that encode the engineered lysins of the present invention, including lysins with fused peptides, such as peptides derived from CeA. Such nucleotides can encode various lysins, including, for example, LysAB2, PlyAB1, PlyE146, 68 Lysin, and ABgp46 linker peptides, and antimicrobial peptides. In certain embodiments, the nucleic acid molecules can comprise SEQ ID NO: 14, SEQ ID NO: 15, SEQ ID NO: 16, SEQ ID NO: 17, and SEQ ID NO: 18 or at least 80%, 85%, 90%, 95%, 98%, or 99% sequence identity to a SEQ ID NO: 14, SEQ ID NO: 15, SEQ ID NO: 16, SEQ ID NO: 17, and SEQ ID NO: 18. The nucleic acid molecules can further comprise nucleic acid sequences that encode peptides, peptide linkers, or antimicrobial peptides, such as, for example, SEQ ID NO: 1 and/or SEQ ID NO: 19. In some embodiments, the nucleic acid molecules of the present disclosure encode an active fragment of the lysin or modified lysin disclosed herein. The term “active fragment” refers to a portion of a full-length lysin, which retains one or more biological activities of the reference lysin. Thus, an active fragment of a lysin or modified lysin, as used herein, inhibits the growth, or reduces the population, or kills Gram-negative bacteria in the absence or presence of, or in both the absence and presence of, human serum.

In certain embodiments, the present disclosure is directed to a vector comprising a nucleic acid molecule encoding any of the lysins disclosed herein or a complementary sequence of the presently isolated polynucleotides. In some embodiments, the vector is a plasmid or cosmid. In other embodiments, the vector is a viral vector, wherein additional DNA segments can be ligated into the viral vector. In some embodiments, the vector can autonomously replicate in a host cell into which it is introduced. In some embodiments, the vector can be integrated into the genome of a host cell upon introduction into the host cell and thereby be replicated along with the host genome.

In some embodiments, particular vectors, referred to herein as “recombinant expression vectors” or “expression vectors,” can direct the expression of genes to which they are operatively linked. A polynucleotide sequence is “operatively linked” when it is placed into a functional relationship with another nucleotide sequence. For example, a promoter or regulatory DNA sequence is said to be “operatively linked” to a DNA sequence that codes for an RNA and/or a protein if the two sequences are operatively linked, or situated such that the promoter or regulatory DNA sequence affects the expression level of the coding or structural DNA sequence. Operatively linked DNA sequences are typically, but not necessarily, contiguous.

In some embodiments, the present disclosure is directed to a vector comprising a nucleic acid molecule selected from SEQ ID NO: 14, SEQ ID NO: 15, SEQ ID NO: 16, SEQ ID NO: 17, and SEQ ID NO: 18 that encodes a lysin selected from SEQ ID NO: 9, SEQ ID NO: 10, SEQ ID NO: 11, SEQ ID NO: 12, and SEQ ID NO: 13. The vector can further comprise a nucleic acid sequence that encodes a linker peptide, such as, for example, SEQ ID NO: 19 and an antimicrobial peptide derived from, for example, CeA, such as, for example, SEQ ID NO: 1.

In one embodiment, the subject compositions are formulated as an orally-consumable product, such as, for example a food item, capsule, pill, or drinkable liquid. An orally deliverable pharmaceutical is any physiologically active substance delivered via initial absorption in the gastrointestinal tract or into the mucus membranes of the mouth. The topic compositions can also be formulated as a solution that can be administered via, for example, injection, which includes intravenously, intraperitoneally, intramuscularly, intrathecally, or subcutaneously. In other embodiments, the subject compositions are formulated to be administered via the skin through a patch or directly onto the skin for local or systemic effects. The compositions can be administered sublingually, buccally, rectally, or vaginally. Furthermore, the compositions can be sprayed into the nose for absorption through the nasal membrane, nebulized, inhaled via the mouth or nose, or administered in the eye or ear.

Orally consumable products according to the invention are any preparations or compositions suitable for consumption, for nutrition, for oral hygiene, or for pleasure, and are products intended to be introduced into the human or animal oral cavity, to remain there for a certain period of time, and then either be swallowed (e.g., food ready for consumption or pills) or to be removed from the oral cavity again (e.g., chewing gums or products of oral hygiene or medical mouth washes). While an orally-deliverable pharmaceutical can be formulated into an orally consumable product, and an orally consumable product can comprise an orally deliverable pharmaceutical, the two terms are not meant to be used interchangeably herein.

Orally consumable products include all substances or products intended to be ingested by humans or animals in a processed, semi-processed, or unprocessed state. This also includes substances that are added to orally consumable products (particularly food and pharmaceutical products) during their production, treatment, or processing and intended to be introduced into the human or animal oral cavity.

Orally consumable products can also include substances intended to be swallowed by humans or animals and then digested in an unmodified, prepared, or processed state; the orally consumable products according to the invention therefore also include casings, coatings, or other encapsulations that are intended to be swallowed together with the product or for which swallowing is to be anticipated.

In one embodiment, the orally consumable product is a capsule, pill, syrup, emulsion, or liquid suspension containing a desired orally deliverable substance. In one embodiment, the orally consumable product can comprise an orally deliverable substance in powder form, which can be mixed with water or another liquid to produce a drinkable orally-consumable product.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the adjuvant composition, carrier or excipient use in the subject compositions may be contemplated.

In one embodiment, the composition can be made into aerosol formulations so that, for example, it can be nebulized or inhaled. Suitable pharmaceutical formulations for administration in the form of aerosols or sprays are, for example, powders, particles, solutions, suspensions or emulsions. Formulations for oral or nasal aerosol or inhalation administration may also be formulated with carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

In one embodiment, the composition can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

In one embodiment, the composition can be formulated for administration via topical application onto the skin, for example, as topical compositions, which include rinse, spray, or drop, lotion, gel, ointment, cream, foam, powder, solid, sponge, tape, vapor, paste, tincture, or using a transdermal patch. Suitable formulations of topical applications can comprise in addition to any of the pharmaceutically active carriers, for example, emollients such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants such as glycerin, propylene glycol, polyethylene glycol, sorbitol solution, and 1,2,6 hexanetriol or permeation enhancers such as ethanol, isopropyl alcohol, or oleic acid.

### Methods of Producing Lysin Antimicrobial Agents

In certain embodiments, the present disclosure includes methods for producing lysin polypeptides fused to peptide residues of defensins, including CeA, which kill or inhibit the growth of one or more Gram-negative bacteria. In some embodiments, polynucleotide sequences encoding lysin polypeptides and peptide residues of defensins can be encoded by a system or vector suitable to maintain, propagate or express polynucleotides and/or to express a polypeptide in a host. The appropriate DNA/polynucleotide sequence may be inserted into the expression system by any of a variety of well-known and routine techniques.

A variety of host/expression vector combinations may be employed in expressing the polynucleotide sequences encoding lysin polypeptides of the present disclosure. Large numbers of suitable vectors are known to those of skill in the art, and are commercially available. Such vectors include, for example, chromosomal, episomal and virus-derived vectors, such as, for example, vectors derived from bacterial plasmids, from bacteriophages, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. Furthermore, said vectors may provide for the constitutive or inducible expression of lysin polypeptides of the present disclosure. More specifically, suitable vectors include but are not limited to derivatives of SV40 and known bacterial plasmids, such as, for example, E. coli plasmids colE1, pCR1, pBR322, pMB9, pET and their derivatives, plasmids such as RP4, pBAD24 and pBAD-TOPO; phage DNAS, such as, for example, the numerous derivatives of phage X, such as, for example, NM989, and other phage DNA, such as, for example, M13 and filamentous single stranded phage DNA; yeast plasmids; vectors useful in eukaryotic cells, such as, for example, vectors useful in insect or mammalian cells; vectors derived from combinations of plasmids and phage DNAs, such as plasmids that have been modified to employ phage DNA or other expression control sequences; and the like.

In another embodiment, the present disclosure is directed to a host cell comprising any of the vectors disclosed herein including the expression vectors comprising the polynucleotide sequences encoding the lysins of the present disclosure. A wide variety of host cells are useful in expressing the present polypeptides. Non-limiting examples of host cells suitable for expression of the present polypeptides include eukaryotic and prokaryotic hosts, such as, for example strains of E. coli, Pseudomonas, Bacillus, Streptomyces, fungi (e.g., Saccharomyces cerevisiae and Pichia pastoris), and animal cells, such as, for example, CHO, Rl.l, B-W and L-M cells, African Green Monkey kidney cells (e.g., COS 1, COS 7, BSC1, BSC40, and BMT10), insect cells (e.g., Sf9), and human cells and plant cells in tissue culture. While the expression host may be any known expression host cell, in a typical embodiment the expression host is one of the strains of E. coli. These include, but are not limited to commercially available E. coli strains such as Top 10 (ThermoFisher Scientific, Inc., Waltham, Mass.), DH5a (Thermo Fisher Scientific, Inc.), XLI-Blue (Agilent Technologies, Inc., Santa Clara, Calif.), SCS 110 (Agilent Technologies, Inc.), JM109 (Promega, Inc., Madison, Wis.), LMG194 (ATCC), and BL21 (Thermo Fisher Scientific, Inc.).

### Methods of Treating Bacterial Infections

In one embodiment, the present disclosure provides methods for treatment of a bacterial infection in a subject caused by Gram-negative bacteria comprising administering to the subject an effective amount of a lysin polypeptide having at least 80%, at least 85%, at least 90%, at least 95% amino acid sequence identity to SEQ ID NO: 9, SEQ ID NO: 10, SEQ ID NO: 11, SEQ ID NO: 12, and SEQ ID NO: 13 fused to the amino acid of SEQ ID NO: 1.

In certain embodiments, compositions of the subject invention to be administered to subject may depend on a number of factors such as the activity of infection being treated such as, for example, the age, health and general physical condition of the subject to be treated or the activity of a particular lysin. In certain embodiments, effective amounts of the lysin to be administered may fall within the range of about 1 mcg/ml to about 150 mcg/ml. In certain embodiments, the lysin may be administered 1-4 times daily for a period ranging from 1 to 14 days.

It is contemplated that the lysins disclosed herein may provide a rapid bactericidal and, when used in sub-MIC amounts, may provide a bacteriostatic effect. It is further contemplated that the lysins disclosed herein may be active against a range of antibiotic-resistant bacteria and may not be associated with evolving resistance. Based on the present disclosure, in a clinical setting, the present lysins may be a potent alternative (or additive) for treating infections arising from drug- and multidrug-resistant bacteria alone or together with antibiotics (including antibiotics to which resistance has developed). It is believed that existing resistance mechanisms for Gram-negative bacteria do not affect sensitivity to the lytic activity of the present lysins.

In certain embodiments, the present lysins may be used for the control, disruption, and treatment of bacterial biofilm formed by Gram-negative bacteria. Biofilm formation occurs when microbial cells adhere to each other and are embedded in a matrix of extracellular polymeric substance (EPS) on a surface. The growth of microbes in such a protected environment that is enriched with biomacromolecules (e.g. polysaccharides, nucleic acids and proteins) and nutrients allow for enhanced microbial cross-talk and increased virulence. Biofilm may develop in any supporting environment including living and nonliving surfaces such as, for example, the mucus plugs of the CF lung, contaminated catheters, and contact lenses.

The terms “infection” and “bacterial infection” are meant to include respiratory tract infections (RTIs), such as respiratory tract infections in patients having cystic fibrosis (CF), lower respiratory tract infections, such as acute exacerbation of chronic bronchitis (ACEB), acute sinusitis, community-acquired pneumonia (CAP), hospital-acquired pneumonia (HAP) and nosocomial respiratory tract infections; sexually transmitted diseases, such as, for example gonococcal cervicitis and gonococcal urethritis; urinary tract infections; acute otitis media; sepsis, including, for example, neonatal septisemia and catheter-related sepsis; and osteomyelitis. Infections caused by drug-resistant bacteria and multidrug-resistant bacteria are also contemplated. Non-limiting examples of infections caused by Gram-negative bacteria include: nosocomial infections such as, for example, respiratory tract infections especially in cystic fibrosis patients and mechanically-ventilated patients, bacteremia and sepsis, wound infections, particularly those of burn victims and those with atopic dermatitis (eczema), urinary tract infections post-surgery infections on invasive devises, endocarditis by intravenous administration of contaminated drug solutions, infections in patients with acquired immunodeficiency syndrome, cancer chemotherapy, steroid therapy, hematological malignancies, organ transplantation, renal replacement therapy, and other conditions with severe neutropenia; community-acquired infections such as, for example, community-acquired respiratory tract infections, meningitis, folliculitis and infections of the ear canal caused by contaminated water, malignant otitis externa in the elderly and diabetics, osteomyelitis of the calcaneus in children, ye infections commonly associated with contaminated contact lens, skin infections such as nail infections in people whose hands are frequently exposed to water, gastrointestinal tract infections, and musculoskeletal system infections.

In some embodiments, inhibiting the growth, or reducing the population, or killing at least one species of Gram-negative bacteria comprises contacting bacteria with the lysins as described herein, wherein the bacteria are present on a surface of such as, for example, medical devices, floors, stairs, walls and countertops in hospitals and other health related or public use buildings and surfaces of equipment in operating rooms, emergency rooms, hospital rooms, clinics, and bathrooms. Examples of medical devices that can be protected using the lysins described herein include but are not limited to tubing and other surface medical devices, such as, for example, urinary catheters, mucous extraction catheters, suction catheters, umbilical cannulae, contact lenses, intrauterine devices, intravaginal and intraintestinal devices, endotracheal tubes, bronchoscopes, dental prostheses and orthodontic devices, surgical instruments, dental instruments, tubings, dental water lines, fabrics, paper, indicator strips (e.g., paper indicator strips or plastic indicator strips), adhesives (e.g., hydrogel adhesives, hot-melt adhesives, or solvent-based adhesives), bandages, tissue dressings or healing devices and occlusive patches, and any other surface devices used in the medical field. The devices may include electrodes, external prostheses, fixation tapes, compression bandages, and monitors of various types. Medical devices can also include any device which can be placed at the insertion or implantation site such as the skin near the insertion or implantation site, and which can include at least one surface which is susceptible to colonization by Gram-negative bacteria.

In certain embodiments, inhibiting the growth, or reducing the population, or killing at least one species of Gram-negative bacteria comprises contacting bacteria with the lysins as described herein, wherein the bacteria are present on a surface of or in livestock such as, for example, cows, pigs, goats, chickens, sheep, rabbit, guinea big, camel, llama, honey bees, fish or in places where livestock reside, such as, for example, livestock feed, water sources for livestock, stalls, transportation vehicles, and livestock bedding.

### Materials and Methods

### Bacterial Strains and Culture Condition

All bacterial strains used in this study are listed in Table 1. A multidrug-resistant strain of A. baumannii, MDR-AB2, isolated from the sputum samples of a patient with pneumonia at PLA Hospital 307 was supplied by the Beijing Institute of Microbiology and Epidemiology [42]. Other bacterial strains were acquired either from the American Type Culture Collection or from Bioresource Collection and Research Center of Taiwan. Clinical isolates of various bacteria were kindly provided by the Beijing Institute of Microbiology and Epidemiology and Prince of Wales Hospital, Hong Kong. All the bacterial strains were grown in Nutrient Broth (NB) medium at 37° C.

### Plasmids Construction

All plasmids were constructed using standard cloning methods. Genes encoded for four globular lysins (LysAB2 (SEQ ID NO: 9), PlyAB1 (SEQ ID NO: 10), PlyE146 (SEQ ID NO: 11) 68 lysin (SEQ ID NO: 12), and ABgp46 (SEQ ID NO: 13), Table 2) were synthesized with BamHI, HindIII and XhoI sites. The synthetic nucleic acid molecules were cloned into the pET-28a(+) expression vector (Novagen, Merck KGaA, Darmstadt, Germany) using BamHI and XhoI sites. For C-terminal peptide modified lysins, the genes encoding for CeA peptide residues 1-8 (KWKLFKKI (SEQ ID NO: 1)) were attached to the C-terminus of the globular endolysin with GGSGG linkers by annealing two synthesized primers and subcloning using HindIII and XhoI sites. For N-terminal modifications, NdeI and BamHI sites were used. For the E55A mutation of LysAB2, the mutated gene was amplified using overlapping PCR from constructed LysAB2-KWK plasmid and cloned into a pET-28a(+) plasmid. Primers (SEQ ID NO: 21-28) used in this work, were listed in Table 3. The sequence of the peptide engineered endolysin was confirmed via DNA sequencing.

### Recombinant Proteins Expression and Purification

Protein Expression

All constructed plasmids were transformed into E. coli BL21 (DE3) cells and colonies were grown overnight at 37° C. in LB media supplemented with 50 μg/mL kanamycin. The start culture was grown overnight, and then it was used to inoculate LB media supplemented with antibiotics at 1:100 ratio. The cell culture was grown at 37° C. to reach an OD₆₀₀ of ˜0.6 before 0.25 mM IPTG was added to induce protein expression. After grown at 37° C. for 3 h, cells were harvested for protein purification.

Purification of Lysins

All enzymes were purified by nickel affinity chromatography using HisTrap™ HP column (GE Healthcare). Briefly, harvested cells were re-suspended in lysis buffer containing 10 mM imidazole, 50 mM phosphate/300 mM sodium chloride (pH 8.0). The cell suspension was lysed by sonication and centrifuged. The supernatant was collected, filtered, and loaded into the column. The bound protein was eluted by imidazole gradient from 10 mM to 500 mM. Pure protein fractions eluted with imidazole gradient were collected and exchanged with PBS (pH 7.4). The purity of the protein was analyzed by 12% SDS-PAGE and all the proteins were at least 90% pure. After purification, all proteins were flash frozen under liquid nitrogen and stored at −80° C.

### Antibacterial Activity Assay

Logarithmic phase bacteria were prepared by inoculating overnight culture at 1:100 ratio in NB media and then shaking 180 rpm for 3-4 hours to reach around OD₆₀₀ 0.6 at 37° C. And the stationary phase bacteria were cultured for overnight (OD₆₀₀ 1.2-1.4). Then the cells were centrifuged and washed once with PBS buffer and resuspended in PBS to an OD₆₀₀ of 0.6. The bacterial suspension was then diluted 100 times with PBS (around 10⁶ cfu/mL) and mixed with different concentrations of the corresponding enzymes or PBS buffer 37° C. for 2 hours. The treated bacteria were then serially diluted and plated for colony counts. For time killing assay, 2 μM, 4 μM, 8 μM, 16 μM of LysAB2-KWK lysins were incubated with logarithmic phase MDR-AB2 bacteria (around 10⁶ cfu/mL), samples were withdrawn at 15 min, 30 min, 60 min, and 120 min for counting of viable bacterial cells. For bacterial spectrum test, all Gram-negative and Gram-positive bacteria were cultured to logarithmic phase and then bacteria (around 10⁶ cfu/mL) were treated with 8 μM native lysins or modified lysins at 37° C. for 2 h followed by plating for bacterial counts. All assays were performed in triplicate and repeated at least in two independent experiments.

### Scanning Electron Microscopy (SEM)

Scanning Electron Microscopy (SEM) was conducted described as previously [43]. Logarithmic phase A. baumannii bacteria were washed twice with and resuspended in PBS buffer at OD600=0.6. Then approximately 10′ cells were incubated at room temperature with 8 μM LysAB2 and LysAB2 KWK for 15 min or 2 h. Cells were then fixed with 2.5% (v/v) glutaraldehyde at 4° C. overnight. Thereafter, the fixed cells were washed twice with PBS and dehydrated with a graded ethanol series (15%, 30%, 50%, 70%, 85%, and 100% for twice). The bacterial suspensions were spotted on a polycarbonate membrane filter (GTTP 0.2 μm, Millipore) and dried with vacuum. Finally, the samples were coated with gold and observed with Quanta 400F SEM (FEI).

### Muralytic Assay

A treatment with chloroform/buffer was used to disrupt the membranes of the Gram-negative bacteria, based on the method of Nakimbugwe [37]. Briefly, Logarithmic phase cultures of A. baumannii were centrifuged (3800×g, 5 min, room temperature), the pellets were resuspended in the same volume of chloroform saturated 50 mM Tris buffer (pH 7.7), shaken for 45 min at 25° C. and then centrifuged again (4000×g, 10 min, 4° C.). The resulting cell pellets were washed twice with PBS buffer and finally resuspended in PBS buffer again at an OD₆₀₀ between 0.9 and 1, after which the enzymes (100 ng/ml) were added, respectively. Absorbance was measured at the wavelength 600 nm by a microplate reader (CLARIOstar, BMI Labtech, Germany). Three independent biological replicates were performed for each condition.

### Outer Membrane Permeability Assay

For the investigation of outer membrane permeability, 1-N-phenylnaphthylamine (NPN) uptake assay was performed [40]. NPN is an uncharged, hydrophobic fluorescent probe that has very weak fluorescence in an aqueous environment. However, it shows strong fluorescence in a hydrophobic interior of a membrane. Upon outer membrane disruption, NPN can reach the hydrophobic environment of the membrane, emitting bright fluorescence. First the NPN uptake assay was set up using polymyxin B, which was usually served as positive control. A. baumannii cells were grown to mid-log phase (OD600 0.6-0.8), centrifuged, and resuspended in PBS. Then NPN was added to the final concentration at 10 μM and incubated with varying concentrations (0.625 to 10 μg/mL) of polymyxin B for 5 min. Then the 8 μM different enzymes were incubated with 10⁷ cfu/mL cells in presence of NPN. The fluorescence intensities were recorded using a microplate reader (CLARIOstar, BMI Labtech, Germany) with 350±7.5 nm for excitation and 420±10 nm for emission.

### Antibiofilm Activity Assay

A. baumannii strains were grown in NB medium overnight at 37° C. with continuous shaking 180 rpm. The overnight bacterial culture was diluted with fresh NB medium to a final density of OD₆₀₀=0.2. To initiate the biofilm growth, diluted culture was aliquoted into a 96-well plate at 100 μL/well (Costar, Corning Incorporated, U.S.A) and incubated at 37° C. for 24 h at 100 rpm. Biofilm was washed twice with PBS and treated with PBS (control), LysAB2-KWK, LysAB2 at 150 μL/well and then put the plate at 37° C. for 48 h at 100 rpm. At the end of the incubation time, all medium were removed and the wells were stained with 200 μL 0.1% (w/v) crystal violet for 1 hour. After staining, the crystal violet solution was removed and the wells were washed with 200 μL PBS for three times. Then, 200 μL of 70% ethanol was added to dissolve the crystal violet and 100 μL solution was transferred to a new plate for quantification of the residual biofilm biomass using a microplate reader (CLARIOstar, BMI Labtech, Germany) at 570 nm. Three independent biological replicates were performed for each condition.

### Antibacterial Activity in Human Serum

To test the endolysin antibacterial activity in human serum, A. baumannii cells in log phase were washed once and resuspend in PBS buffer. Then, cells (around 10⁶ cfu/mL) were treated with 8 μM enzymes or PBS buffer in the presence of 1%-5% human serum (Sigma-Aldrich, Shanghai, China) at 37° C. for 2 h, respectively. The viable cell numbers were evaluated by plating on LA plates. Kill assays were done in triplicate and repeated at least two independent experiments.

### Cytotoxicity of Lysins Against BEAS-2B Cell

BEAS-2B (Human Normal Lung Epithelial Cells) cells were cultured in DMEM (Gibco) containing 10% FBS (Gibco) under standard conditions in a humidified incubator with 5% CO₂ at 37° C. The cytotoxic effect of the lysins on BESA-2B cells was measured by Cell Counting Kit-8. For this, the cells were seeded at density of 10⁴ cells/well in a 96-well plate containing 200 μL of culture medium and incubated for 24 h. Next, the cells were incubated with 0-20 μM lysins for 12 h. Then, 10 μL of WST-8 solution (Beyotime, Shanghai, China) was added to each well and cells were incubated for 2 h at 37° C. Absorbance was measured at a wavelength of 450 nm using a microplate reader (Multiskan Sky, Thermo Fisher). The PBS group was served as a negative control. Three independent biological replicates were performed for each condition.

TABLE 1
Description of the bacterial strains used in this study.

| Strain | Description and Characteristics | Origin |
|---|---|---|
| A. baumannii MDR-AB2 | Multidrug-resistant Gram-negative strain; host for phage IME-AB2 | Clinical isolate from hospitcal |
| E. coli Top10 | Laboratory strain for cloning use | Novagen, USA |
| E. coli BL21 | Laboratory strain for protein expression | Novagen, USA |
| E. coli MG1655 | Laboratory strain; Gram-negative | Novagen, USA |
| P. aeruginosa ATCC 27853 | Gram-negative reference strain | ATCC, USA |
| P. aeruginosa PAO1 | Multidrug-resistant Gram-negative strain | ATCC, USA |
| P. aeruginosa PAV237 | Multidrug-resistant Gram-negative strain | Clinical isolate from hospitcal |
| A. baumannii ATCC 19606 | Gram-negative reference strain | ATCC, USA |
| A. baumannii M3237 | Multidrug-resistant Gram-negative strain | Clinical isolate, BCRC 80276 |
| A. baumannii #1 | Multidrug-resistant Gram-negative strain | Clinical isolate from hospitcal |
| A. baumannii #2 | Multidrug-resistant Gram-negative strain, A. baumannii 126 | Clinical isolate from hospitcal |
| A. baumannii #3 | Multidrug-resistant Gram-negative strain, A. baumannii 690 | Clinical isolate from hospitcal |
| A. baumannii #4 | Multidrug-resistant Gram-negative strain, A. baumannii IMPR | Clinical isolate from hospitcal |
| K. pneumoniae 501 | Multidrug-resistant Gram-negative strain | Clinical isolate from hospitcal |
| S. aureus ATCC 25923 | Gram-postive reference strain | ATCC, USA |
| E. faecium 19 | Gram-positive strain | Clinical isolate from hospitcal |
| E. faecium 20 | Gram-positive strain | Clinical isolate from hospitcal |

TABLE 2
Description of the endolysins used in this study.

| Name | Sequence ID | Reference |
|---|---|---|
| LysAB2 | HM755898.1 | [1] |
| PlyAB1 | NC_021316.1, M172_gp50 | [2] |
| PlyE146 | EKK47578.1 | [3] |
| 68 Lysin | NC_041857.1, FDG67_gp68 | [4] |

TABLE 3
Description of the primers
used in this study.

| Name | Sequence (5′-3′) | Usage |
|---|---|---|
| NKWK- | TATGAAATGGAAACT | N-terminal |
| NdeI-Fw | GTTCAAGAAAATCGG | |
| | TTCCG | |
| | (SEQ ID NO: 21) | |
| NKWK- | GATCCGGAACCGATT | modification |
| BamHI-Rv | TTCTTGAACAGTTTC | |
| | CATTTCA | |
| | (SEQ ID NO: 22) | |
| CKWK- | AGCTTGGTGGCTCTG | C-terminal |
| HindIII- | GTGGCAAATGGAAAC | |
| Fw | TGTTCAAGAAAATCT | |
| | GAC | |
| | (SEQ ID NO: 23) | |
| CKWK- | TCGAGTCAGATTTTC | modification |
| XhoI-Rv | TTGAACAGTTTCCAT | |
| | TTGCCACCAGAGCCA | |
| | CCA | |
| | (SEQ ID NO: 24) | |
| AB2KWK- | CGCGGATCCATGATT | LysAB2 |
| BamHI-Fw | CTGACTAAAGAC | |
| | ((SEQ ID NO: 25) | |
| AB2KWK- | GTGCTCGAGTCAGAT | mutation |
| XhoI-Rv | TTTCTTGAACAG | |
| | (SEQ ID NO: 26) | |
| 55A-Fw | CAAGTGGCTATCGAA | |
| | CTATGCAACCTATTA | |
| | AAGAAGCTGGTTCTG | |
| | ATAG | |
| | (SEQ ID NO: 27) | |
| 55A-Rv | GCATAGTTCGATAGC | |
| | CACTTGGTAAACCAG | |
| | TCGCATGGTAAATAG | |
| | TAGC | |
| | (SEQ ID NO: 28) | |

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLES

### Example 1-Engineering the C-Terminus of LysAB2

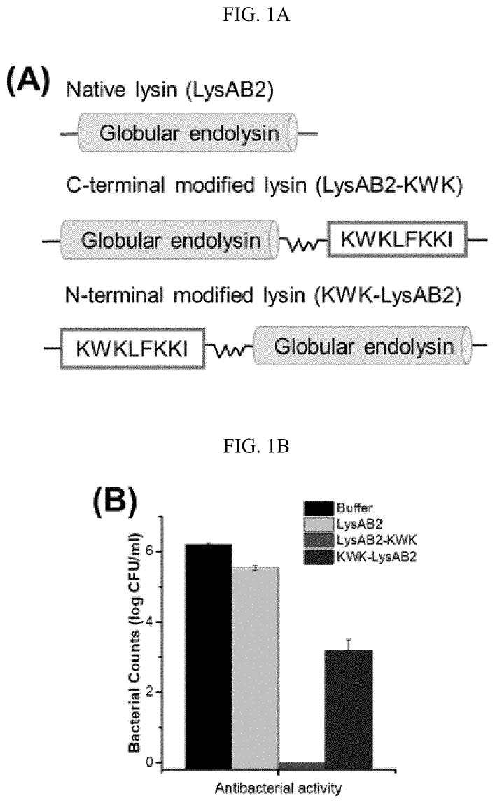

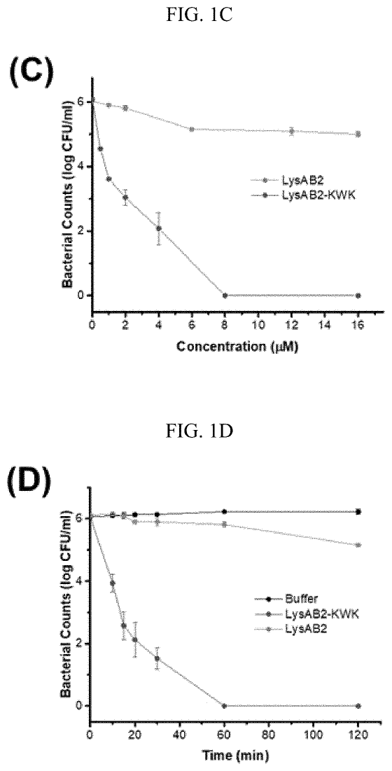

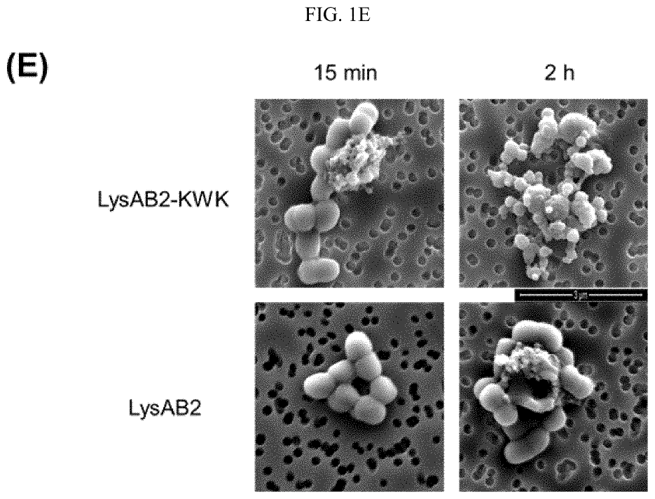

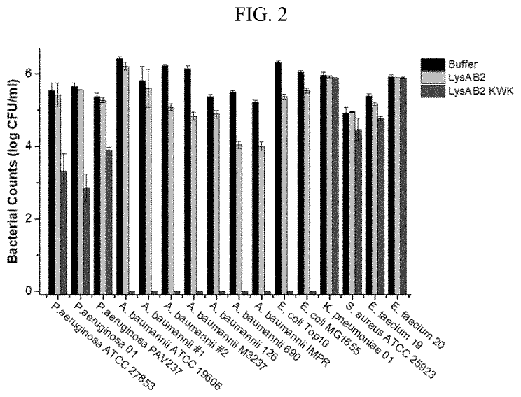

LysAB2 has only modest antibacterial activity that it could cause up to 2-log reduction of the bacterial counts at a concentration of 20 μM, but it was active against a number of Gram-negative and Gram-positive bacteria, including A. baumannii, Escherichia co/i and Streptococcus sanguis [20]. The positively-charged CeA peptide residues 1-8, KWKLFKKI (SEQ ID NO: 1), has been reported to enhance the antibacterial activity of a modular lysin [19]. Two modified LysAB2 constructs were obtained by fusing the CeA peptide octamer at either the C-terminus or the N-terminus of LysAB2 to give an N-terminal fusion construct (KWK-LysAB2) and a C-terminal fusion construct (LysAB2-KWK) (FIG. 1A). Both proteins were expressed and purified, and their antibacterial activities against a multidrug resistant A. baumannii strain, MDR-AB2, at the log phase were compared together with the native lysin. LysAB2-KWK at a concentration of 8 μM completely eradicated the bacterial culture, whereas the native LysAB2 only caused <1 log reduction and the KWK-LysAB2 resulted in a 3-log bacterial reduction under the same conditions (FIG. 1B). This result indicates that fusion with the CeA peptide converts LysAB2 from a modest antibacterial enzyme to a highly potent one, and the C-terminal modification was superior to the N-terminal modification.

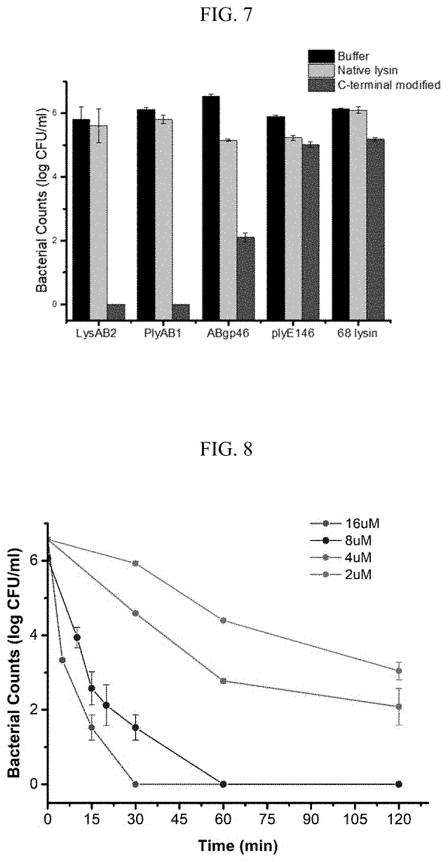

LysAB2-KWK showed a dose-dependent antibacterial activity against the log phase A. baumannii cell culture (FIG. 1C). No viable A. baumannii cells could be detected when the concentration of LysAB2-KWK reached 8 μM. A 3-log reduction in cell numbers was observed at a LysAB2-KWK concentration as low as 2 μM, which was still more effective than the native LysAB2 at 16 μM. We then tracked the bacterial counts of the viable A. baumannii cells at different time points at a LysAB2-KWK concentration of 8 μM. A 4-log reduction was seen within the first 15 min, and the bacteria were completely killed within 1 h (FIG. 1D). The fast killing kinetics are also shown to be dose-dependent: complete cell lytic response could be accomplished within 30 min at 16 μM (FIG. 8). Cell surface morphological changes in response to the native LysAB2 and modified LysAB2-KWK enzymes were observed under scanning electron microscopy (SEM). A 15-min treatment with LysAB2-KWK (8 μM) caused marked change on bacterial cells, while most of the LysAB2-treated cells remained unchanged. A 2 h treatment with LysAB2-KWK caused cell lysis in almost all the cells (FIG. 1E). Taken together, LysAB2-KWK was established to be a fast and effective antibacterial enzyme against the drug-resistant A. baumannii.

The activity spectrum of the native lysin and modified LysAB2-KWK was tested against a panel of 13 Gram-negative bacteria and 3 Gram-positive bacteria (FIG. 2). The antimicrobial activity of LysAB2-KWK was strain specific but retained a broad spectrum antimicrobial activity against A. baumannii, E. coli and P. aeruginosa, including the multi-drug resistant isolates. LysAB2-KWK was strongly active against all A. baumannii isolates tested, causing a complete elimination for all A. baumannii isolates at a concentration of 8 μM. And three tested P. aeruginosa isolates were all susceptible to LysAB2-KWK with a reduction that ranged from 1.5 to 2.8-log decrease in viable bacterial cell counts. Compared to the engineered PlyA, the modular lysin modified with the same CeA peptide sequence, LysAB2-KWK, showed a higher potency against E. coli; it caused a complete eradication of 10⁶ cfu/ml cells at a concentration 8 μM [19]. No activity was observed against K. pneumoniae. The variable susceptibility of these Gram-negative clinical isolates may be due to their different OM structure. For Gram-positive strains, which do not have an outer membrane outside the peptidoglycan layer, no enhanced activity was found against Staphylococcus aureus or Enterococcus faecium strains when comparing LysAB2-KWK to LysAB2.

### Example 2-Antibacterial Activity Towards Stationary Phase and The Biofilm of A. baumannii

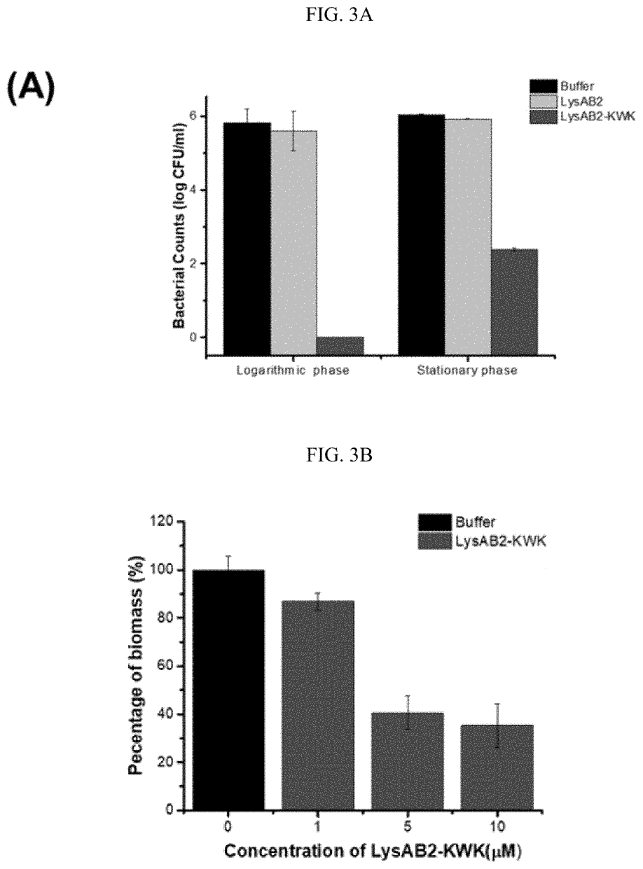

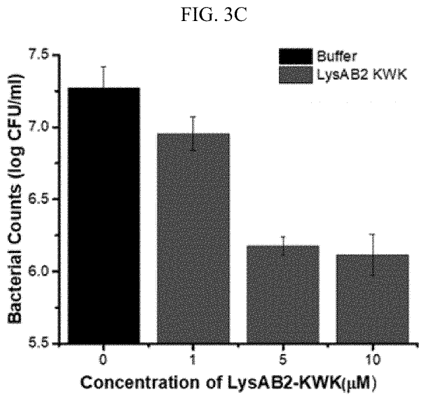

The antibacterial activity of the modified lysin against MDR-AB2 at different growth phases was examined. Many lysins showed different activities to bacterial cells at different growth phases and often have a lower activity against bacteria in stationary phase than in towards bacteria in log phase [19, 22, 24, 25]. LysAB2-KWK showed a pronounced antibacterial activity against A. baumannii at stationary phase: a 4-log reduction towards the stationary phase bacteria (from 6 log to 2.1 log) at a concentration 10 μM of LysAB2-KWK, a concentration at which the log phase A. baumannii were completely eradicated (FIG. 3A). A. baumannii in the log phase are therefore more sensitive than those in the stationary phase to LysAB2-KWK, suggesting that the MDR-AB2 bacterial cells at these two growth phases have different surface compositions leading to different outer membrane penetration capacities. Our results were consistent with previous discoveries but showed a higher potency in the absence of OMPs. The potent activity against the stationary phase therefore establishes that LysAB2-KWK has a high versatility towards bacterial cells at different states.

We also tested the antibiofilm activity of LysAB2-KWK against A. baumannii as biofilm formation retards the bactericidal effect of antibiotics and contributes to the development of antibiotic resistance [26,27]. Briefly, biofilms were developed for 24 h and then treated with different concentrations of LysAB2-KWK and PBS, as a negative control, for 10 h at 37° C. The residual biofilm was then quantified by crystal violet (CV) staining and viable cell counting [28]. According to the results of the CV staining assay (FIG. 3B), the biomass could be disrupted to 40.6%±6.9% after incubating with 5 μM enzyme, but further increasing the enzyme concentration to 10 μM did not result in a further reduction in the residual biomass of the biofilm. The results agreed with the viable cell counting in which 5 μM LysAB2-KWK resulted in a 1-log reduction (from 7.29±0.17 to 6.17±0.06 log) and no further bacterial reduction was noted in the biofilm treated with a concentration of 10 μM of enzyme (FIG. 3C). Nonetheless, our results confirmed the capability of LysAB2-KWK in degrading an already-formed A. baumannii biofilm.

### Example 3-Serum Activity, Cytotoxicity and Storage Stability

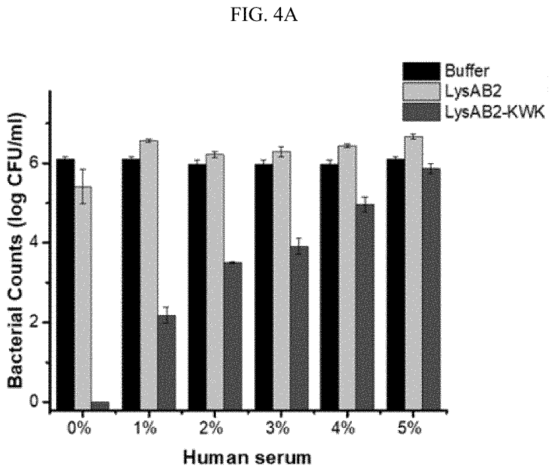

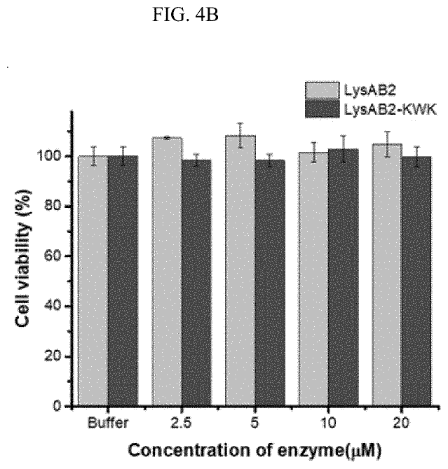

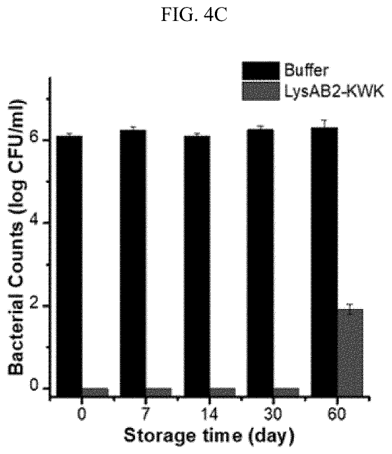

The practical use of the antibacterial enzymes has requirements beyond antimicrobial activity, such as, for example, serum activity, cytotoxicity towards human cells, and storage stability. Intolerance to serum is well documented for lysins with intrinsic outer membrane penetrating capabilities, significantly limiting their clinical applicability [19,29,30,31]. A hypothesis is that the existence of negatively charged molecules in the serum neutralize the positive charges in the C-terminals of the lysins, resulting in activity loss [19]. To fully evaluate the therapeutic potential of the modified LysAB2-KWK, we tested its activity against A. baumannii in the presence of human serum. Interestingly, the native LysAB2 was completely inhibited in the presence of 1% serum, but LysAB2-KWK could retain some of its activity in a buffer containing up to 4% serum despite the positively charged CeA peptide (FIG. 4A). These findings suggest that the application of LysAB2-KWK would have to be limited to infections in a low serum environment. While topical application certainly avoids the encounter of serum, the treatment of lung infection via the inhalation route may still be feasible because the lung contains a low serum level. Raz et al. established a lung infection model and demonstrated that intratracheally administered PlyPa91 lysin, which could also retain certain antibacterial activity in a serum level of 4%, could protect mice from fatal lung infection, with a 70% rescue rate [30].

The cytotoxicity of LysAB2-KWK was also evaluated using the Cell Counting Kit-8 (CKK8) kit [32]. BESA-2B cells (normal human lung bronchial epithelial cell) were incubated with various concentrations of lysins for 12 h, and cells were subjected to the CKK8 kit to quantify the numbers using the absorbance of 570 nm. The result shows that no cytotoxicity against BESA-2B cells was detected even at a high dose of 20 μM (FIG. 4B), suggesting LysAB2-KWK could be a safe treatment.

To develop lysins as commercially viable biopharmaceuticals, ensuring their stability upon storage, transportation, and end use are critical. We, therefore, evaluated the storage stability of LysAB2-KWK at 4° C. The antibacterial activity against the logarithmic growth phase A. baumannii was measured at day 7, 14, 30 and 60. Results showed that LysAB2-KWK was stable for up to 1 month of storage without any activity loss, whereas 2 months of storage resulted in partial loss of activity (FIG. 4C). Further formulation designs will be needed to improve the storage stability of LysAB2-KWK [33-36].

### Example 4-Mechanism of the Enhanced Antibacterial Activity

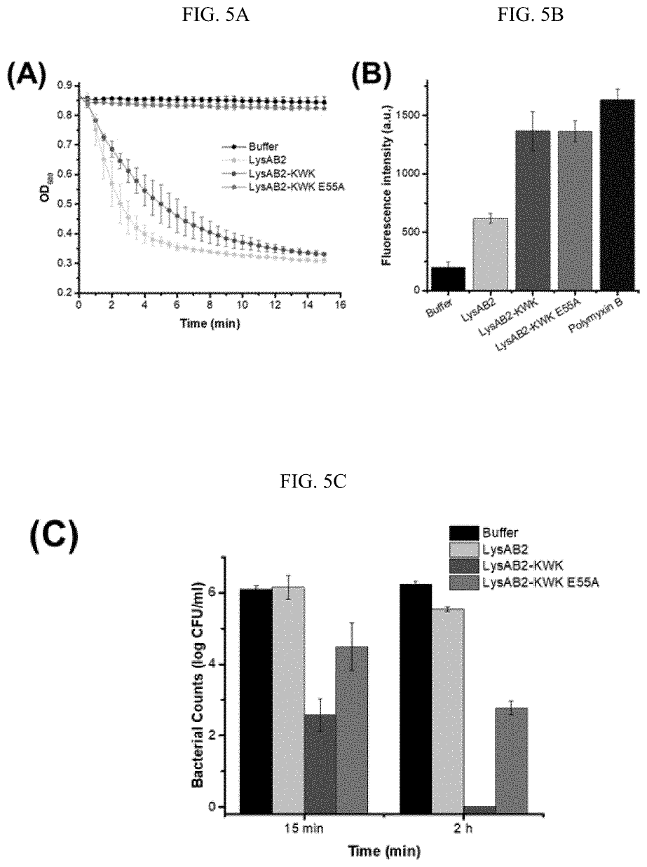

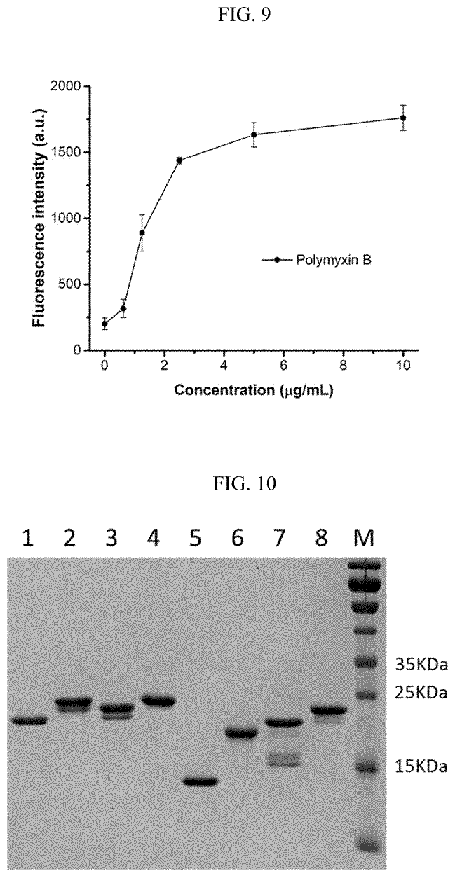

Although it is hypothesized that the extended positively charge peptide can enhance the outer membrane penetration of modified lysins to improve the bacterial killing efficiency, no experimental evidence was available in the literature. Therefore, the underlying mechanisms responsible for the superior antibacterial activity of LysAB2-KWK were investigated in detail. Frist, we determined whether the CeA peptide fusion could affect the activity of PG degradation using a muralytic assay. Briefly, bacteria were treated with a chloroform-saturated Tris buffer to remove the outer membrane and expose the PG layer as a substrate to the enzymes [37,38]. LysAB2-KWK and LysAB2 showed similar rates in decreasing the turbidity of the outer membrane-removed cells (FIG. 5A). This indicates that peptide-fusion did not enhance or deteriorate the intrinsic PG degrading activity. Next, we used 1-N-phenylnaphthylamine (NPN) uptake assay to determine the outer membrane permeability in the presence of different enzymes. Using the fluorescent molecule NPN as an indicator, the destabilization of OM outer membrane can be measured by the fluorescence signal enhancement due to the enrichment of NPN in the hydrophobic membrane [39,40]. LysAB2-KWK treatment significantly increased the fluorescent intensity as compared with LysAB2 at a concentration of 8 μM (FIG. 5B), establishing that the KWK tag significantly increased the outer membrane permeability. According to fluorescence intensity of NPN, the outer membrane permeabilization activity of LysAB2-KWK was equivalent to polymyxin B, a well-known antimicrobial peptide against Gram-negative bacteria (FIG. 5B and FIG. 9) [41]. We also generated a loss-of-function mutant by mutating the glutamic acid at 55 position to alanine (E55A), as E55 was predicted to be the catalytic residue in LysAB2 [20]. The E55A mutation lost almost all the muralytic activity (FIG. 5A), while maintaining the outer permeabilization activity of LysAB2-KWK (FIG. 5B). These results indicate that the C-terminal peptide fusion increased the outer membrane permeability of LysAB2, and the muralytic activity and the outer membrane permeabilization activity are independent. Interestingly, the E55A mutant lysin showed partial antibacterial activity, causing a 3.3-log reduction of the log-phase A. baumannii cells at a concentration of 8 μM (FIG. 5C), despite the loss of the PG degrading activity. This suggested that the OM permeabilization could also cause bactericidal effect. Altogether, these experiments dissected the muralytic activity and the outer membrane permeabilization activity and showed that the enhanced antibacterial activity of the LysAB2-KWK was solely attributed to the enhanced outer membrane permeability due to the C-terminal modification.

### Example 5-Sequence Dependence of the C-Terminal Engineering

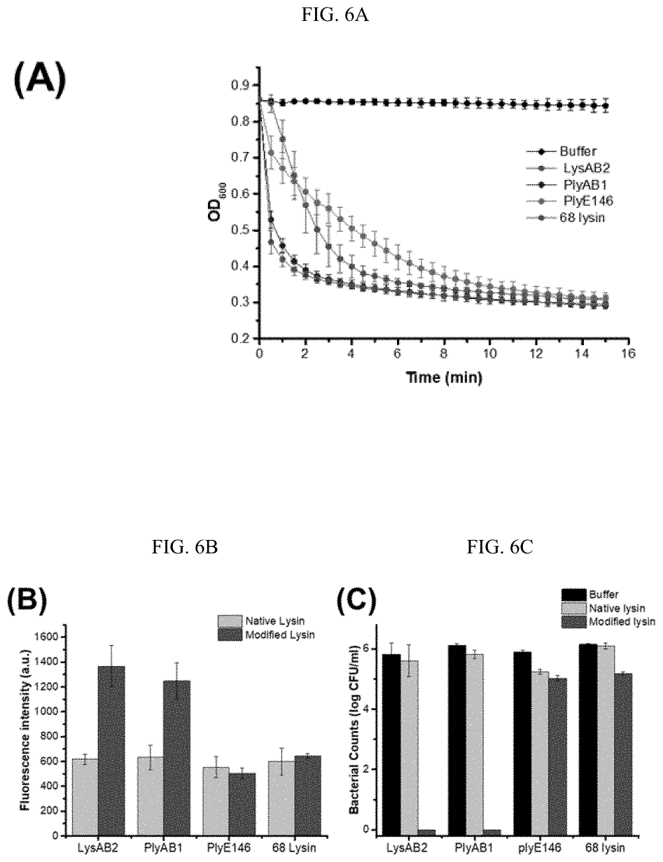

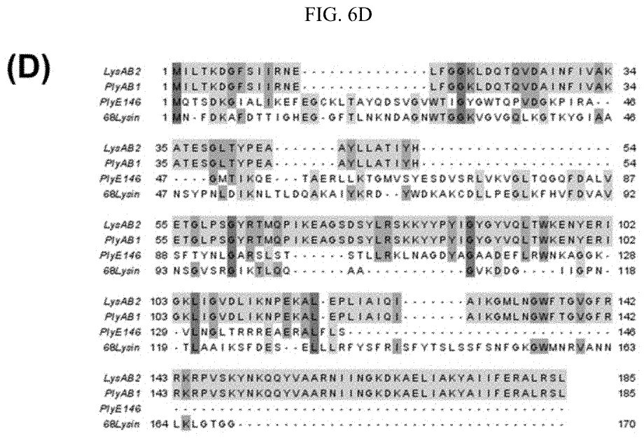

After achieving success with the LysAB2 lysin, we next explored whether the same C-terminal engineering strategy is applicable to other globular lysins. We extended this peptide modification to other two published globular lysins, PlyAB1 (NC_021316.1) [21] PlyE146 (EKK47578.1) [29] with modest intrinsic antibacterial activity, and an unpublished 68 lysin (NC_041857.1) which is derived from a phage active against MDR-AB2 [42]. Muralytic assays showed that all three lysins could effectively degrade the peptidoglycan layer of A. baumannii (FIG. 6A). Among these three lysins, only the C-terminal engineered PlyAB1-KWK showed similar enhancement as LysAB2-KWK on outer membrane permeability (FIG. 6B) and a higher antibacterial activity than the native PlyAB1 against A. baumannii (FIG. 6C). Contrarily, the C-terminally engineered PlyE146 and 68 lysin achieve no significant enhancement in the outer membrane permeability and hence no difference in the antibacterial activity between the engineered and native enzymes. To further elucidate these observations, we compared the sequences of these lysins and suggested the differences in the outer membrane permeability of the engineered lysins would strongly depend on the C-terminal sequences. PlyAB1, having the same C-terminal sequence as LysAB2, benefited from the peptide modification, whereas the C-terminal sequences of the other two globular lysins which have distinct sequences from LysAB2 showed no improvement (FIG. 6D). We reason that the native C-terminal sequence of LysAB2 and PlyAB1 globular lysins combined with the CeA peptide, with the whole sequence being IIFERALRSLGGSGGKWKLFKKI, provides an optimal outer membrane permeabilizing result.

Bacteriophage lysins are a class of murein hydrolases that can degrade the PG layer of bacteria cells. This substrate, however, is not easily accessible, particularly for Gram-negative bacteria, with which the PG layers are sandwiched between the outer membrane and inner membrane. While lysins rely on an additional protein, holin, to trespass the inner membrane during the phage lytic step, these enzymes are not designed to target the PG layer from outside. As there are no natural transporters for outer membrane permeabilization, engineering lysins to 33 equip them with the outer membrane permeability lies at the center of the development of this class of antibacterial enzymes for potential clinical use. Here we show for the first time that the C-terminus of globular lysins harbor certain outer membrane permeabilization activity, and this activity can be drastically enhanced by appending a CeA peptide at the C-terminal end of a globular lysin. This feature, however, is not generally in all types of lysins. Enhancing the outer membrane permeability through C-terminal engineering allows the murein hydrolytic activity to be fully revealed, shown as an outstanding antibacterial activity towards a range of Gram-negative bacteria. On top of this finding, we discovered an engineered LysAB2, LysAB2-KWK, is highly feasible for future development for clinical use. To our knowledge, this work presents the first systematic exploration of the C-terminus of globular lysins and unveils an important step towards the application of antibacterial enzymes.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Piddock L J V. Reflecting on the final report of the O'Neill Review on Antimicrobial Resistance. Lancet Infect Dis. 2016; 16(7):767-768.
2. WHO, Global priority list of antibiotic-resistant bacteria to guide research, discovery, and development of new antibiotics. 2017
3. Breijyeh Z, Jubeh B, Karaman R. Resistance of gram-negative bacteria to current antibacterial agents and approaches to resolve it. Molecules. 2020; 25(6): 1340.
4. Fodor A, Abate B A, Deák P, Fodor L, Gyenge E, Klein M G, Koncz Z, Muvevi J, Ötvös L, Székely G, Vozik D, Makrai L. Multidrug Resistance (MDR) and Collateral Sensitivity in Bacteria, with Special Attention to Genetic and Evolutionary Aspects and to the Perspectives of Antimicrobial Peptides-A Review. Pathogens. 2020; 9(7):522.
5. Fenton M, Ross P, McAuliffe O, O'Mahony J, Coffey A. Recombinant bacteriophage lysins as antibacterials. Bioeng Bugs. 2010; 1(1):9-16
6. Haddad Kashani H, Schmelcher M, Sabzalipoor H, Seyed Hosseini E, Moniri R. Recombinant Endolysins as Potential Therapeutics against Antibiotic-Resistant Staphylococcus aureus: Current Status of Research and Novel Delivery Strategies. Clin Microbiol Rev. 2017; 31(1):e00071-17.
7. Schmelcher M, Donovan D M, Loessner M J. Bacteriophage endolysins as novel antimicrobials. Future Microbiol. 2012; 7(10):1147-1171.
8. Abdelkader K, Gerstmans H, Saafan A, Dishisha T, Briers Y. The Preclinical and Clinical Progress of Bacteriophages and Their Lytic Enzymes: The Parts are Easier than the Whole. Viruses. 2019; 11(2):96.
9. Schirmeier E, Manfred B, Briers Y, Stefan M, Colistin resistant mcr-1-positive Escherichia coli isolates: No resistance development against the highly bactericidal Artilysin® Art-x01, in the Phages 2017 Oxford. 2017: Oxford, England, UK
10. Briers Y, Lavigne R. Breaking barriers: expansion of the use of endolysins as novel antibacterials against Gram-negative bacteria. Future Microbiol. 2015; 10(3):377-90.
11. Ghose, C. and C. W. Euler, Gram-Negative Bacterial Lysins. Antibiotics (Basel), 2020. 9(2).
12. Gutiérrez D, Briers Y. Lysins breaking down the walls of Gram-negative bacteria, no longer a no-go. Curr Opin Biotechnol. 2020; 68:15-22.
13. Vázquez R, García E, Garcia P. Phage Lysins for Fighting Bacterial Respiratory Infections: A New Generation of Antimicrobials. Front Immunol. 2018; 9:2252.
14. Lai W C B, Chen X, Ho M K Y, Xia J, Leung S S Y. Bacteriophage-derived endolysins to target gram-negative bacteria. Int J Pharm. 2020; 30; 589:119833.
15. Briers Y, Walmagh M, Van Puyenbroeck V, et al. Engineered endolysin-based “Artilysins” to combat multidrug-resistant gram-negative pathogens. mBio. 2014; 5(4):e01379-14.
16. Briers Y, Walmagh M, Grymonprez B, et al. Art-175 is a highly efficient antibacterial against multidrug-resistant strains and persisters of Pseudomonas aeruginosa.

Antimicrob Agents Chemother. 2014; 58(7):3774-3784
17. Defraine V, Schuermans J, Grymonprez B, Govers S K, Aertsen A, Fauvart M, Michiels J, Lavigne R, Briers Y. Efficacy of Artilysin Art-175 against Resistant and Persistent Acinetobacter baumannii. Antimicrob Agents Chemother. 2016; 60(6):3480-8.
18. Gerstmans H, Criel B, Briers Y. Synthetic biology of modular endolysins.

Biotechnol Adv. 2018; 36(3):624-640.
19. Yang H, Wang M, Yu J, Wei H. Antibacterial Activity of a Novel Peptide-Modified Lysin Against Acinetobacter baumannii and Pseudomonas aeruginosa. Front Microbiol. 2015; 6:1471.
20. Lai M J, Lin N T, Hu A, et al. Antibacterial activity of Acinetobacter baumannii phage ϕAB2 endolysin (LysAB2) against both gram-positive and gram-negative bacteria. Appl Microbiol Biotechnol. 2011; 90(2):529-539.
21. Huang G, Shen X, Gong Y, et al. Antibacterial properties of Acinetobacter baumannii phage Abp1 endolysin (PlyAB1). BMC Infect Dis. 2014; 14:681.
22. Oliveira H, Thiagarajan V, Walmagh M, et al. A thermostable Salmonella phage endolysin, Lys68, with broad bactericidal properties against gram-negative pathogens in presence of weak acids. PLoS One. 2014; 9(10):e108376
23. Lim J A, Shin H, Heu S, Ryu S. Exogenous lytic activity of SPN9CC endolysin against gram-negative bacteria. J Microbiol Biotechnol. 2014; 24(6):803-811.
24. Rolfe M D, Rice C J, Lucchini S, et al. Lag phase is a distinct growth phase that prepares bacteria for exponential growth and involves transient metal accumulation. J Bacteriol. 2012; 194(3):686-701.
25. Typas A, Banzhaf M, Gross C A, Vollmer W. From the regulation of peptidoglycan synthesis to bacterial growth and morphology. Nat Rev Microbiol. 2011; 10(2):123-136.
26. The Role of Bacterial Biofilm in Antibiotic Resistance and Food Contamination. Int J Microbiol. 2020; 25:1705814.
27. Jamal M, Ahmad W, Andleeb S, Jalil F, Imran M, Nawaz M A, Hussain T, Ali M, Rafiq M, Kamil M A. Bacterial biofilm and associated infections. J Chin Med Assoc. 2018; 81(1):7-11.
28. Chang R Y K, Das T, Manos J, Kutter E, Morales S, Chan H K. Bacteriophage PEV20 and Ciprofloxacin Combination Treatment Enhances Removal of Pseudomonas aeruginosa Biofilm Isolated from Cystic Fibrosis and Wound Patients. AAPS J. 2019; 21(3):49.
29. Larpin Y, Oechslin F, Moreillon P, Resch G, Entenza J M, Mancini S. In vitro characterization of PlyE146, a novel phage lysin that targets Gram-negative bacteria. PLoS One. 2018; 13(2):e0192507
30. Raz A, Serrano A, Hernandez A, Euler C W, Fischetti V A. Isolation of Phage Lysins That Effectively Kill Pseudomonas aeruginosa in Mouse Models of Lung and Skin Infection. Antimicrob Agents Chemother. 2019; 63(7):e00024-19.
31. Thandar M, Lood R, Winer B Y, Deutsch D R, Euler C W, Fischetti V A. Novel Engineered Peptides of a Phage Lysin as Effective Antimicrobials against Multidrug-Resistant Acinetobacter baumannii. Antimicrob Agents Chemother. 2016; 60(5):2671-9.
32. Lebourgeois S, Fraisse A, Hennechart-Collette C, Guillier L, Perelle S, Martin-Latil S. Development of a Real-Time Cell Analysis (RTCA) Method as a Fast and Accurate Method for Detecting Infectious Particles of the Adapted Strain of Hepatitis A Virus. Front Cell Infect Microbiol.2018; 8:335.
33. Abouhmad A, Dishisha T, Amin M A, Hatti-Kaul R. Immobilization to Positively Charged Cellulose Nanocrystals Enhances the Antibacterial Activity and Stability of Hen Egg White and T4 Lysozyme. Biomacromolecules. 2017; 18(5):1600-1608.
34. Ciepluch K, Skrzyniarz K, Barrios-Gumiel A, et al. Dendronized Silver Nanoparticles as Bacterial Membrane Permeabilizers and Their Interactions With P. aeruginosa Lipopolysaccharides, Lysozymes, and Phage-Derived Endolysins. Front Microbiol. 2019; 10:2771.
35. Rathore N, Rajan R S. Current perspectives on stability of protein drug products during formulation, fill and finish operations. Biotechnol Prog. 2008; 24(3):504-14.
36. Bai J, Yang E, Chang P S, Ryu S. Preparation and characterization of endolysin-containing liposomes and evaluation of their antimicrobial activities against gram-negative bacteria. Enzyme Microb Technol. 2019; 128:40-48
37. Nakimbugwe D, Masschalck B, Deckers D, Callewaert L, Aertsen A, Michiels C W. Cell wall substrate specificity of six different lysozymes and lysozyme inhibitory activity of bacterial extracts. FEMS Microbiol Lett. 2006; 259(1):41-46.
38. Briers Y, Lavigne R, Volckaert G, Hertveldt K. A standardized approach for accurate quantification of murein hydrolase activity in high-throughput assays. J Biochem Biophys Methods. 2007; 70(3):531-533.
39. Loh B, Grant C, Hancock R E. Use of the fluorescent probe 1-N-phenylnaphthylamine to study the interactions of aminoglycoside antibiotics with the outer membrane of Pseudomonas aeruginosa. Antimicrob Agents Chemother. 1984; 26(4):546-551.
40. T. Helander I M, Mattila-Sandholm T. Fluorometric assessment of gram-negative bacterial permeabilization. J Appl Microbiol. 2000; 88(2):213-9.
41. Poirel L, Jayol A, Nordmann P. Polymyxins: Antibacterial Activity, Susceptibility Testing, and Resistance Mechanisms Encoded by Plasmids or Chromosomes. Clin Microbiol Rev. 2017; 30(2):557-596.
42. Peng F, Mi Z, Huang Y, Yuan X, Niu W, Wang Y, Hua Y, Fan H, Bai C, Tong Y. Characterization, sequencing and comparative genomic analysis of vB_AbaM-IME-AB2, a novel lytic bacteriophage that infects multidrug-resistant Acinetobacter baumannii clinical isolates. BMC Microbiol. 2014; 14:181.
43. Farkas A, Maróti G, Kereszt A, Kondorosi É. Comparative Analysis of the Bacterial Membrane Disruption Effect of Two Natural Plant Antimicrobial Peptides. Front Microbiol. 2017; 8:51.
44. Fair R J, Tor Y. Antibiotics and bacterial resistance in the 21st century. Perspect Medicin Chem. 2014; 28(6):25-64.
45. Cooper M A, Shlaes D. Fix the antibiotics pipeline. Nature. 2011; 472(7341):32.
46. Oliveira H, Vilas Boas D, Mesnage S, Kluskens L D, Lavigne R, Sillankorva S, Secundo F, Azeredo J. Structural and Enzymatic Characterization of ABgp46, a Novel Phage Endolysin with Broad Anti-Gram-Negative Bacterial Activity. Front Microbiol. 2016 26; 7:208.

We claim:

1. An isolated polypeptide comprising a globular endolysin fused to a CeA peptide, wherein the CeA peptide consists of SEQ ID NO: 1.

2. The polypeptide of claim 1, wherein the CeA peptide is fused to the C-terminus of the globular endolysin.

3. The polypeptide of claim 1, wherein the globular endolysin is LysAB2 (SEQ ID NO: 9), PlyAB1 (SEQ ID NO: 10), ABgp46 (SEQ ID NO: 13), or a variant thereof having at least 95% identity.

4. The polypeptide of claim 1 comprising a sequence selected from SEQ ID NO: 29, SEQ ID NO: 30, or SEQ ID NO: 31.

5. A recombinant DNA molecule comprising a DNA sequence that encodes a globular endolysin fused to a CeA peptide, wherein the encoded CeA peptide consists of SEQ ID NO: 1.

6. The recombinant DNA of claim 5, wherein the encoded CeA peptide is fused to the C-terminus of the globular endolysin.

7. The recombinant DNA of claim 5, wherein the globular endolysin is LysAB2 (SEQ ID NO: 9), PlyAB1 (SEQ ID NO: 10), ABgp46 (SEQ ID NO: 13), or a variant thereof having at least 95% identity.

8. The recombinant DNA of claim 5, wherein the DNA sequence that encodes the globular endolysin fused to a CeA is expressed by a unicellular organism.

9. The recombinant DNA of claim 5, wherein the unicellular organism is Escherichia coli, Saccharomyces cerevisiae, or Pichia pastoris.

10. A method of inhibiting the growth, reducing the population, inhibiting an infection, or killing of at least one species of Gram-negative bacteria, the method comprising contacting the bacteria with a composition comprising an effective amount of an endolysin polypeptide comprising a globular endolysin fused to a CeA peptide consisting of SEQ ID NO: 1, wherein the endolysin polypeptide has the property of inhibiting the growth of, or reducing an initial population of, or killing at least one species of Gram-negative bacteria.

11. The method of claim 10, wherein the one species of Gram-negative bacteria is selected from the genera consisting of Klebsiella, Enterobacter, Escherichia, Citrobacter, Salmonella, Yersinia, Pseudomonas, Acinetobacter, and Francisella.

12. The method of claim 11, wherein the Gram-negative bacteria from the genera Acinetobacter is Acinetobacter baumannii.

13. The method of claim 10, further comprising administering to a subject diagnosed with at risk for, or exhibiting symptoms of an infection of at least one species of Gram-negative bacteria the composition comprising the effective amount of the endolysin polypeptide comprising the globular endolysin fused to a CeA peptide consisting of SEQ ID NO: 1.

14. The method of claim 10, wherein the composition is a solution, a suspension, an emulsion, an inhalable powder, an aerosol, or a spray.

15. The method of claim 10, wherein the Gram-negative bacterial infection is a topical infection or a systemic pathogenic bacterial infection.

16. The method of claim 10, wherein the Gram-negative bacteria are resistant to at least one antibiotic.

17. The method of claim 10, wherein the Gram-negative bacteria are in a biofilm.

18. The method of claim 10, wherein the Gram-negative bacteria are present in an infection of the skin of the individual.

19. The method of claim 10, wherein the Gram-negative bacteria are present in an infection of lung of the individual.

20. The method of claim 10, wherein the Gram-negative bacteria are in contact with sera of the individual.
